(19) United States
(12) Patent Application Publication
(10) Pub. No.: US 2025/0101364 A1
(43) Pub. Date: Mar. 27, 2025

(54) ELECTROCHEMICAL CO2 RELEASE AND PHOTOSYNTHETIC STORAGE

(71) Applicant: Battelle Memorial Institute, Richland, WA (US)

(72) Inventors: Chinmayee V. Subban, Seattle, WA (US); Scott J. Edmundson, Sequim, WA (US); Christopher R. Myers, Spokane, WA (US); Charles F. Hibbeln, Port Angeles, WA (US)

(73) Assignee: Battelle Memorial Institute, Richland, WA (US)

(21) Appl. No.: 18/898,275

(22) Filed: Sep. 26, 2024

Related U.S. Application Data
(60) Provisional application No. 63/540,852, filed on Sep. 27, 2023.

(51) Int. Cl. C12M 1/00; B01D 61/44; B01D 61/50; C02F 1/469; C02F 103/08; C12M 1/34

(52) U.S. Cl. CPC C12M 43/00; B01D 61/445; B01D 61/50; C02F 1/4693; C12M 41/06; C12M 41/26; C02F 2103/08

(57) ABSTRACT

Disclosed herein are aspects of a system for coupling electrochemical marine carbon capture with photosynthesis. In some aspects of the present disclosure, the system comprises an electrochemical cell that converts saline water into (i) a base stream, (ii) an at least partially deionized water stream, and (iii) an acid stream. In some aspects, the system further comprises a biomass cultivation unit in fluid communication with the acid stream, the biomass cultivation unit comprising a photosynthetic organism. The acid stream catalyzes release of CO₂ in a growth medium for the photosynthetic organism to accelerate growth of the photosynthetic organism relative to growth without the acid stream and facilitates CO₂ storage by the photosynthetic organism. Also disclosed herein are aspects of a method for coupling electrochemical marine carbon capture with photosynthesis.

### CROSS-REFERENCE TO RELATED APPLICATION

This application claims the benefit of, and priority to, U.S. Provisional Application No. 63/540,852, filed Sep. 27, 2023, which is incorporated herein by reference in its entirety.

### ACKNOWLEDGMENT OF GOVERNMENT SUPPORT

This invention was made with government support under Contract DE-AC0576RL01830 awarded by the U.S. Department of Energy. The government has certain rights in the invention.

### FIELD

The present disclosure concerns systems and methods for carbon dioxide removal (CDR).

### BACKGROUND

Oceans are the planet's largest carbon sink, and when carbon dioxide (CO₂) reacts with seawater it forms carbonic acid and subsequently H⁺ and bicarbonate. The increased seawater acidity has direct impacts on coastal industry, communities, and ecosystems. Reversal of ocean acidification is a primary co-benefit of implementing marine carbon dioxide removal (mCDR) strategies for decarbonization.

mCDR interventions alter the ocean's CO₂-bicarbonate-carbonate equilibrium in favor of lowering ocean acidity. mCDR is a topic of growing interest both within governmental and private-sector entities. Unlike terrestrial CDR, mCDR does not need land or freshwater and offers ecosystem co-benefits of ocean deacidification and restoration. The ability to test mCDR methods at-scale or under field-relevant conditions to allow reliable environmental impact assessments has presented issues in developing any relevant industrial application of this technology. There exists a need in the art for new methods that facilitate carrying out mCDR approaches at impactful levels.

### SUMMARY

Disclosed aspects of the present disclosure advantageously provide a system for coupling electrochemical marine carbon capture with photosynthesis. In some aspects of the present disclosure, the system comprises an electrochemical cell configured to be in fluid communication with a saline water source comprising saline water (e.g., seawater or brackish water), wherein the electrochemical cell converts the saline water into (i) a base stream comprising hydroxide ions, (ii) an at least partially deionized water stream, and (iii) an acid stream comprising hydrogen ions. In some aspects, the system further comprises a biomass cultivation unit in fluid communication with the acid stream produced by the electrochemical cell, the biomass cultivation unit comprising a photosynthetic organism and a growth medium for the photosynthetic organism, and wherein the acid stream catalyzes release of CO₂ from the growth medium to accelerate growth of the photosynthetic organism relative to growth without the acid stream and facilitates CO₂ storage by the photosynthetic organism.

Other disclosed aspects of the present disclosure advantageously provide a method for coupling electrochemical marine carbon capture with photosynthesis. In some aspects, the method comprises using an electrochemical cell configured to be in fluid communication with a saline water source comprising saline water (e.g., seawater or brackish water), wherein the electrochemical cell converts the saline water into (i) a base stream comprising hydroxide ions, (ii) an at least partially deionized water stream, and (iii) an acid stream comprising hydrogen ions. In some aspects, the method further comprises inputting the acid stream into a biomass cultivation unit comprising a photosynthetic organism and a growth medium for the photosynthetic organism, wherein the acid stream catalyzes CO₂ release from the growth medium to accelerate growth of the photosynthetic organism relative to growth without the acid stream and facilitates CO₂ storage by the photosynthetic organism.

The foregoing and other objects, features, and advantages of the present disclosure will become more apparent from the following detailed description, which proceeds with reference to the accompanying figures.

### BRIEF DESCRIPTION OF THE DRAWINGS

FIG. 1 shows aspects of a system for coupling electrochemical marine carbon capture with photosynthesis according to aspects of the present disclosure.

FIG. 2 shows aspects of an example of an electrochemical cell that can be used in the system of FIG. 1.

FIG. 3 shows aspects of another system for coupling electrochemical marine carbon capture with photosynthesis according to aspects of the present disclosure.

FIG. 4 shows a flow diagram depicting aspects of an exemplary method for coupling electrochemical marine carbon capture with photosynthesis according to aspects of the present disclosure.

FIG. 5 shows a schematic diagram of an exemplary computing system according to aspects of the present disclosure.

FIG. 6 shows a plot of measured AFDW:OD₇₅₀ ratio across multiple acid addition tests.

FIG. 7 shows a simplified process flow diagram for acid generation with BPMED, wherein (i) all three streams (acid, base, and seawater) circulate in a closed loop starting with a source of filtered seawater, (ii) inline pH and conductivity sensors for each stream are used to record changes to water quality before and after BPMED, and (iii) a laptop was used to control the power supply, record current/voltage data, and sensor readings.

FIG. 8 is a plot of measured in-line pH of the acid stream of FIG. 7 in the first 15 minutes of BPMED unit operation at different current values.

FIG. 9 is a plot comparing energy and time for an acid stream to reach pH 3 at different current densities using a method and system according to aspects of the present disclosure.

FIG. 10 shows a photograph of a bench-scale closed system set-up comprising exemplary algae growth chambers (referred to herein as photobioreactors or PBRs) to prevent air-sea gas exchange, wherein the inset shows an illustration of Picochlorum celeri cultures.

FIG. 11 is a plot of culture pH following different volumes of acid addition, wherein the data are extracted from full time series of culture pH values during a given acid addition evaluation; the inset is a time plot of an example acid addition, showing initial pH overshoot and eventual settling.

FIG. 12 is a plot showing algal productivity enhancement from acid additions at the end of an evaluation, wherein the productivity is measured as ash-free dry weight (AFDW) and is reported as daily productivity for replicate evaluations; error bars are standard deviation of these replicates and AFDW as presented here is an inferred multiple of OD₇₅₀ measurement.

FIG. 13 is a plot of the pH response to the acid additions summarized in FIG. 11.

FIG. 14 is a plot of a relationship between seawater pCO₂ and seawater pH, wherein atmospheric pCO₂ is indicated by a solid line for reference.

FIG. 15 is a plot comparing algal growth between single and multiple acid additions.

FIG. 16 shows quartile box plots of culture pH over the course of the evaluation window.

FIG. 17 is a simplified diagram of a model system used to analyze CO₂ footprints for two BPMED-acid use cases: (1) transportation to a point of use 100 miles away, and (2) cultivation and processing of algae, and transportation of the resulting algal product over 100 miles; the 0.0129 kg_{CO2} generated via transport in (1) is more than suppressed by the retention of 29.9 kg_{CO2} via BPMED effluent acid-enhanced algae growth.

FIG. 18 shows a plot of Tetraselmis suecica (or “T. suecica”) UTEX2286 growth (represented by OD₇₅₀) in sealed flasks after adding 0 vol. %, 0.2 vol. %, or 0.4 vol. % acid; cultures were diluted on the 5th day along with the addition of more acid.

FIG. 19 shows a plot of pH in the T. suecica UTEX2286 cultures described in FIG. 18.

FIG. 20 is a simplified diagram of a continuous-flow bioreactor configured for automated addition of acid according to aspects of the present disclosure.

FIG. 21 is a plot of average growth (n=2) of T. suecica UTEX2286 with 0 vol. %, 0.2 vol. %, and 0.4 vol. % acid addition after air sparging (100 mL/min) for four days following acid addition and sealed incubation; inset: (from left to right) cultures with 0 vol. %, 0.2 vol. %, and 0.4 vol. % acid addition after sealed incubation and air sparging and error bars show one standard deviation.

FIG. 22 is a plot of CO₂ concentration in headspace of a sealed flask after the addition of 0.4 vol. % acid to either a T. suecica UTEX2286 culture or to abiotic growth media.

FIG. 23 is a plot of average (n=2) OD₇₅₀ for 100-mL T. suecica cultures under alternating conditions of sealed incubation with acid addition, air sparging (100 mL/min), and dilution for three consecutive cycles (starting day 0, 4, and 6) over one week; error bars show one standard deviation.

FIG. 24 is a plot of average (n=2) OD₆₈₀/OD₇₅₀ for 100-mL T. suecica cultures under alternating conditions of sealed incubation with acid addition, air sparging (100 mL/min), and dilution for three consecutive cycles (starting day 0, 4, and 6) over one week; error bars show one standard deviation.

FIG. 25 is a plot of average (n=2) pH for 100-mL T. suecica cultures under alternating conditions of sealed incubation with acid addition, air sparging (100 mL/min), and dilution for three consecutive cycles (starting day 0, 4, and 6) over one week; error bars show one standard deviation.

FIG. 26 is a plot of AFDW for T. suecica cultures grown by alternating sealed incubation with acid addition, air sparging, and dilution for one week with error bars representing variability in replicate AFDW measurements.

### DETAILED DESCRIPTION

### I. Abbreviations

AEM: anion exchange membrane
AFDW: ash-free dry weight
ASIC: application-specific integrated circuit
BPM: bipolar membrane
BPMED: bipolar membrane electrodialysis
CAs: carbonic anhydrases
CCM: carbon concentrating mechanism
CEM: cation exchange membrane
cm: centimeter
CO₂: carbon dioxide
CO₃²⁻: carbonate ion
CPU: central processing unit
DIC: dissolved inorganic carbon
DRAM: dynamic random-access memory
E_{avg}=average voltage
EEPROM: electrically erasable programmable read-only memory
GHG: greenhouse gas
GREET: Greenhouse Gases, Regulated Emissions, and Energy Use in Transportation
Gt: gigaton
H+ ions: hydrogen cations, also referred to as protons
HCl: hydrochloric acid
HCO₃⁻: bicarbonate ion
H₂SO₄: sulfuric acid
hr: hour
Hz: Hertz
i=current
IDE: integrated development environment
kg: kilogram
kWh: kilowatt hour
L: liter
LED: light-emitting diode
mCDR: marine carbon dioxide removal
M: molar
m: meter
mA: milliamp
meq: milliequivalent
min: minute
mg: milligram
mM: millimolar
mL: milliliter
NaOH: sodium hydroxide
N: nitrogen
mN: millinormal
NVRAM: non-volatile random-access memory
OD: optical density
OD₆₈₀: OD at a wavelength of 680 nm
OD₇₅₀: OD at a wavelength of 750 nm
OH⁻ ions: hydroxide anions
P: phosphorus
P. celeri: Picochlorum celeri (a species of algae)
PBR: photobioreactor
pCO₂: partial pressure of CO₂
Pt/IR-MMO: platinum/iridium mixed metal oxide
Q: volumetric flow rate
RAM: random-access memory
RF: radio frequency
ROM: read-only memory
RPM: rotations per minute
RuBisCO: ribulose-1,5-bisphosphate carboxylase/oxygenase
s: second
SRAM: static random-access memory
tₒₙ: time operating
Ti: titanium
μm: micrometer
μmol: micromole
V: Volt
vol. %: percent by volume
vₜₒₜₐₗ=sample volume

### II. Overview of Terms, Ranges, and Definitions

Both the foregoing general description and the following detailed description are exemplary and explanatory only and are not intended to limit the scope of the present disclosure.

As used herein, the use of the singular includes the plural unless specifically stated otherwise. For example, the singular forms “a,” “an” and “the” as used in the specification also include plural aspects unless the context dictates otherwise. Similarly, any singular term used in the specification also means plural or vice versa, unless the context dictates otherwise.

In some examples, values, procedures, or devices may be referred to as “lowest,” “best,” “minimum,” or the like. It will be appreciated that such descriptions are intended to indicate that a selection among many used functional alternatives can be made, and such selections need not be better, smaller, or otherwise preferable to other selections.

Unless explained otherwise, all technical and scientific terms used herein have the same meaning as commonly understood to one of ordinary skill in the art to which this disclosure belongs. Although methods and materials similar or equivalent to those described herein can be used in the practice or testing of the present disclosure, suitable methods and materials are described below. The materials, methods, and examples are illustrative only and not intended to be limiting, unless otherwise indicated. Unless explained otherwise, method steps and components of systems or apparatuses represented by dashed boxes and/or lines in the figures are optional. Other features of the disclosure are apparent from the following detailed description and the claims.

Unless otherwise indicated, all numbers expressing quantities of components, molecular weights, percentages, temperatures, times, and so forth, as used in the specification or claims are to be understood as being modified by the term “about.” Accordingly, unless otherwise indicated, implicitly or explicitly, the numerical parameters set forth are approximations that can depend on the desired properties sought and/or limits of detection under standard test conditions/methods. When directly and explicitly distinguishing embodiments from discussed prior art, the embodiment numbers are not approximates unless the word “about” is recited. Furthermore, not all alternatives recited herein are equivalents.

The term “or combinations thereof” as used herein refers to all permutations and combinations of the listed items preceding the term. For example, “A, B, C, or combinations thereof” is intended to include at least: A, B, C, AB, AC, BC, or ABC, and if order is important in a particular context, also BA, CA, CB, ACB, CBA, BCA, BAC, or CAB. Continuing with this example, expressly included are combinations that contain repeats of one or more item or term, such as BB, AAA, AAB, BBC, AAABCCCC, CBBAAA, CABABB, and so forth.

For the sake of presentation, the detailed description uses terms like “determine” and “use” in some contexts to describe computer operations in a computing system. In the context of a computing system, these terms are high-level abstractions for operations performed by a computer, and should not be confused with acts performed by a human being. The actual computer operations corresponding to these terms vary depending on implementation.
Acid stream: a stream comprising hydrogen ions, produced by an electrochemical cell.
Acidification of growth medium: a process of increasing a concentration of hydrogen ions and reducing a pH of the growth medium using an acid stream.
Batch transport: the process of moving at least a portion of an acid stream to a biomass cultivation unit in one or more batches.
Biomass cultivation unit: a system or apparatus in fluid communication with an acid stream and containing a photosynthetic organism.
Bipolar membrane electrodialysis system (BPMED): an electrochemical cell comprising one or more membranes, which is used to convert a saline water source into base and acid streams.
Brackish water: saline water that has a lower concentration of dissolved salt than seawater, but a greater concentration of dissolved salt than fresh water.
Controller: a device or system that manages, commands, directs, or regulates the behavior of other devices or systems. In some aspects, the controller receives input data, processes it, and generates output signals to control the operation of one or more connected components or systems. In some aspects, the controller monitors and titrates the acid stream to maintain a predetermined pH or pH program in a growth medium.
Electrochemical cell: a device configured to be in fluid communication with a saline water source, and to convert the saline water source into a base stream with hydroxide ions and an acid stream with hydrogen ions.
Fluid communication: the connection between a biomass cultivation unit and an electrochemical cell, allowing the transfer of base and acid streams between the two.
Fluid coupling: the process or state of fluidly connecting an acid stream with a biomass cultivation unit, or an apparatus used to achieve a connection of the acid stream to the biomass cultivation unit.
Growth medium: a substance in a biomass cultivation unit according to aspects of the disclosure in which a photosynthetic organism grows.
Membrane: a selective barrier that allows certain ions or molecules to pass through it while blocking others. In some aspects, the membrane is used in an electrochemical process to facilitate the separation, transport, or conversion of chemical species.
pH program: a dynamic sequence of pH values that a system can target over time. In some aspects, the system can follow a predefined schedule or set of instructions to adjust the pH at different stages of a process.
pH sensor: a sensor that measures the pH of a growth medium.
Photosynthetic organism: an organism, such as an algal or plant species, that uses photosynthesis to grow and capture CO₂.
Potential: an amount of electric potential energy per unit charge at a specific point in an electric field. In some aspects, the potential is expressed in Volts (V).
Predetermined: a value, quantity or parameter that is established or set in advance. In some aspects, the predetermined value, quantity or parameter is based on calculations, standards, or other specifications. In some aspects, this value, quantity or parameter is used as a reference or target in various processes, systems, or operations.
Purification system: a system configured to remove one or more impurities from an input. In some aspects, the purification system is used to pre-treat saline water source, including a water filtration system to filter saline water (e.g., seawater or brackish water) before it enters an electrochemical cell according to aspects of the present disclosure.
Saline water source: an origin of saline water (e.g., seawater or brackish water) that is supplied to an electrochemical cell according to aspects of the present disclosure.
Saline water: water that includes salt. In some aspects, saline water comprises seawater or brackish water according to aspects of the present disclosure.
Titration of acid stream: a process of adding an acid stream to a growth medium in a controlled amount and/or flow rate.

### III. Introduction

Marine carbon dioxide removal (mCDR) can enhance ocean alkalinity using electrochemical methods powered by renewable energy sources. Generally, electrochemical systems produce an acid and base stream from seawater and electricity. In some instances, electrochemical mCDR approaches use a bipolar membrane electrodialysis (BPMED) system, which generates acid, base, and at least partially deionized product streams using seawater and electricity. Either CO₂ is stripped and captured from the acidified stream (direct CO₂ capture), or the basic stream is returned to the ocean for alkalinity enhancement (indirect CO₂ capture). Both approaches face challenges for sustainable deployment, in that they require development of complementary processes. In some aspects, the acid stream is a waste product. It is estimated that ˜1.04 Gt of dry HCl waste is generated per Gt of CO₂ captured by BPMED. Considering the large-scale of mCDR necessary to make meaningful impact on atmospheric CO₂, the acid storage, neutralization, and disposal processes used to balance the system can have prohibitive costs and carbon footprints.

To address these issues and other drawbacks associated with conventional electrolysis, the present disclosure provides a system for coupling electrochemical marine carbon capture with photosynthesis. In some aspects, the system comprises an electrochemical cell configured to be in fluid communication with a saline water source comprising saline water. The electrochemical cell converts the saline water into (i) a base stream comprising hydroxide ions, (ii) an at least partially deionized water stream, and (iii) an acid stream comprising hydrogen ions. In some aspects, the acid stream is input into a biomass cultivation unit comprising a photosynthetic organism and a growth medium for the photosynthetic organism. The acid stream catalyzes CO₂ release from the growth medium to accelerate growth of the photosynthetic organism relative to growth without the acid stream and facilitates CO₂ storage by the photosynthetic organism. Photosynthetic organisms can be significantly more productive if CO₂ is readily available and altering the carbonate-bicarbonate equilibrium in the saline water can make more CO₂ readily available for photosynthetic uptake. By down-shifting the carbonate-bicarbonate equilibrium to more favorable levels of CO₂ and bicarbonate by acid addition, rates of marine photosynthesis can be substantially increased. In some aspects, using the acid can increase marine photosynthetic CO₂ capture rates by 3.5×. Furthermore, coupling the electrochemical ocean alkalinity enhancement (e.g., 1 mol NaOH captures 1 mol CO₂) with marine photosynthesis in acidified saline water (e.g., 1 mol HCl captures 1 mol CO₂) can double the overall amount of CO₂ captured by the system.

Photosynthesis-based marine CO₂ capture is a scalable, sustainable, and readily deployable solution with an existing industry and market. Marine photosynthesis products can be transformed into food, feed, fertilizers, bioplastics, fuels etc., providing an economic incentive for carbon capture. According to aspects of the present disclosure, marine CO₂ capture can occur on-site where electrochemical mCDR is located to provide a productive use for the acid stream and can be easily implemented and scaled-up.

### IV. System

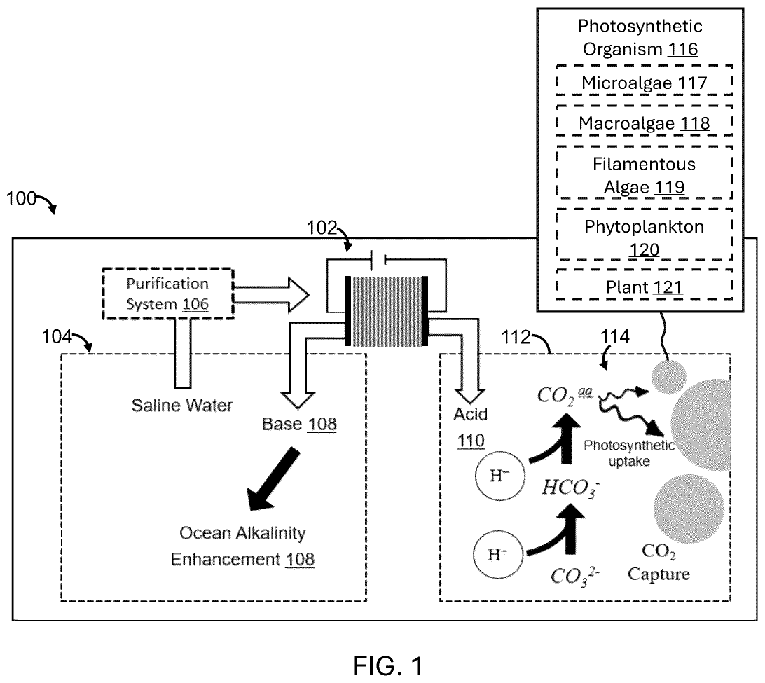

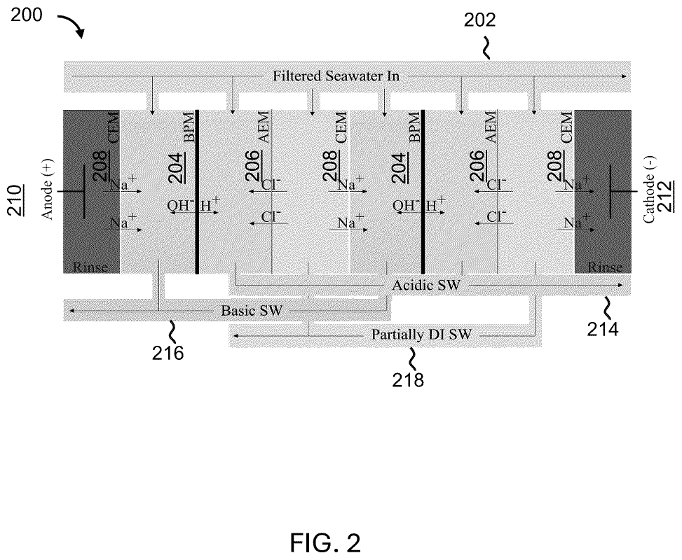

FIG. 1 shows a simplified diagram of a system 100 for coupling electrochemical marine carbon capture with photosynthesis according to aspects of the present disclosure. The system 100 comprises an electrochemical cell 102. In some aspects, the electrochemical cell 102 is in fluid communication with a saline water source 104. In some aspects, the saline water source 104 comprises a source of saline water (e.g., seawater or brackish water). As described in more detail below, the electrochemical cell 102 converts the saline water source into (i) a base stream 108 comprising hydroxide ions and (ii) an acid stream 110 comprising hydrogen ions. In some aspects, a stream of at least partially deionized saline water (e.g., deionized seawater or brackish water) is also produced (as described in more detail below with reference to FIG. 2).

In some aspects, the saline water source 104 comprises unfiltered seawater. In some aspects, the system 100 further comprises a purification system 106, such as a water filtration system or a reverse osmosis system, configured to pre-treat the seawater before the seawater enters the electrochemical cell. This removes impurities from the unfiltered seawater that could clog or degrade the electrochemical cell 102 over time.

In some aspects, the electrochemical cell 102 comprises a BPMED system. A simplified diagram of an exemplary BPMED system 200 is shown in FIG. 2. The BPMED system is a membrane separation system that performs electrodialysis on an input stream 202 using one or more bipolar membranes (BPMs) 204 sandwiched between an anion exchange membrane (AEM) 206 and a cation exchange membrane (CEM) 208. An electric field is applied between the AEM 206 and the CEM 208.

In some aspects, the BPMED system is operated using a constant potential mode or a constant current mode. The constant current mode refers to operating the BPMED system with a fixed electrical current. This mode can maintain a steady rate of ion transport across the membranes, which facilitates efficient separation of ions and production of acids and bases. In contrast, the constant potential mode refers to operating the BPMED system with a fixed voltage rather than a fixed current. This mode can help in maintaining a steady electric field, which helps in the dissociation of water molecules into hydrogen ions (H⁺) and hydroxide ions (OH⁻) at the BPM interface. The steady electric field can also lead to more consistent ion separation and can be useful in processes where the concentration of ions and/or resistance of the solution varies. Operating at a constant potential can also help in optimizing the energy consumption of the BPMED system. It can also provide better control over the process, including in scenarios where the resistance of the solution and/or the concentration of ions changes over time, such as in natural saline water sources.

Current and voltage values are determined by the system size and configuration, and desired output product (e.g., acid or base) quality. In some aspects, the electric field is provided by applying a potential of 0.1 V to 15 V between anode 210 and cathode 212. In some aspects, the potential ranges from 1 V to 3 V. This potential can drive water splitting at the BPMs 204. Operating at higher voltages can increase a rate of ion transport, but also may, in some instances, lead to higher energy consumption and membrane degradation over time. In some aspects, any other suitable potential can be used. Other examples of suitable potentials include those less than 0.1 V and those greater than 10 V.

As described above, the potential applied to the electrochemical cell drives water splitting at the BPMs 204. This potential also draws H⁺ ions towards AEMs 206, where the anions are concentrated, making this side of the BPMs 204 more acidic. Solution is drawn from space(s) between the BPM 204 and the AEM 206, which forms an acid stream 214. OH-ions move towards the CEM, making this side of the BPM 204 more basic. Solution also is drawn from space(s) between the BPM 204 and the CEM 208, which forms a base stream 216. In some aspects, the base stream 216 is returned to the saline water source. In this manner, the BPMED system 200 increases ocean alkalinity and encourages indirect CO₂ removal from the atmosphere. In some aspects, the base stream 216 is, additionally or alternatively, used for one or more alkaline reactions. A remaining stream of at least partially deionized saline water (e.g., deionized seawater or brackish water) 218 can also be returned to the ocean. In some aspects, and as described in more detail below, the acid stream 214 is used to acidify a photosynthetic organism growth medium. The acidification of the growth medium accelerates growth of the photosynthetic organism and facilitates CO₂ capture by the photosynthetic organism.

Referring again to FIG. 1, the acid stream 110 is input into a biomass cultivation unit 112. In some aspects, at least a portion of the acid stream 110 can be transported to the biomass cultivation unit 112 in one or more batches prior to being input into the biomass cultivation unit 112. For example, acid can be shipped (e.g., by truck, rail, or sea, or any other means) from an acid production site to the biomass cultivation unit 112. In this manner, the biomass cultivation unit 112 can serve as a hub or core resource to remediate one or more remote acid sources. In some aspects, the acid stream 110 is fluidly coupled to the biomass cultivation unit 112. For example, the acid stream 110 can be output from the electrochemical cell 102 directly into the biomass cultivation unit 112. This can mitigate financial costs and carbon emissions associated with transporting the acid.

With continued reference to FIG. 1, in some aspects, the biomass cultivation unit 112 comprises a growth medium 114 and a photosynthetic organism 116. The growth medium 114 is saline water-based (e.g., brackish water-based or seawater-based). In some aspects, the growth medium 114 includes added growth-enhancing nutrients, such as sources of nitrogen (N), potassium (K), phosphorous (P), iron (Fe), manganese (Mn), zinc (Zn), or a combination thereof. In some aspects, the photosynthetic organism 116 comprises a microalgal species 117, a macroalgal species 118, a filamentous algal species 119, a phytoplankton species 120, a plant species 121, or a combination thereof. Some examples of suitable photosynthetic organisms include alga (e), such as Picochlorum, Tetraselmis, Chlamydomonas, Tisochrysis, Pavlova, Arthrospira, Spirulina, Galdieria, Limnospira, Asparagopsis, Prionitus, Ulva, Gracilaria, Saccharina, Kappaphycus, Palmeria, and Porphyra, photosynthetic plant(s) such as Zostera marina, and cyanobacteria such as Cyanobacterium. The photosynthetic organism 116 facilitates CO₂ capture and storage through photosynthetic carbon fixation. This activity also facilitates the ability to valorize the acid stream from the electrochemical cell described herein, versus disposing of the acid stream as a waste product.

In some aspects, the photosynthetic organism not only tolerates the presence of acid, but the acid can accelerate photosynthetic organism growth, thereby facilitating greater rates of CO₂ capture. Many microalgae can consume multiple dissolved inorganic carbon species (e.g., CO₂ and/or HCO₃⁻) for photosynthesis, but there is an energy penalty for some carbon species. When CO₂ is absent, the active carbon concentrating mechanism (CCM) comprising enzyme RuBisCO coupled with carbonic anhydrases (Cas) can make more CO₂ from HCO₃⁻; this is energetically more expensive than passive CO₂ diffusion. Altering the CO₂/HCO₃⁻/CO₃²⁻ equilibrium in seawater or brackish water to make CO₂ readily available can modulate the CCM and alter the energy budget of the cell, thereby altering growth and photosynthetic activity. Prior studies have shown enhanced photosynthesis and growth rates through CCM down regulation, but the exact gains can be species-specific. Even when a species is able to use HCO₃⁻, it reaches photosynthetic saturation at much lower concentrations of HCO₃⁻ than if CO₂ is available-confirming that species can be carbon limited in seawater or brackish water. Addition of the BPMED-derived acid to seawater or brackish water can make more CO₂ more readily available to substantially enhance rates of marine photosynthesis.

In some aspects, the growth medium 114 has a pH ranging from 2 to 11. It will also be appreciated that, in some aspects, any other suitable pH range can be used. For example, a lower pH range (e.g., a pH of 1 to 6) can be used for an acidophile. In some aspects, the photosynthetic organism may prefer a more basic environment (e.g. a pH of 10 to 11). In some aspects, the pH can be selected based upon the photosynthetic organism. For example, in some aspects, the growth medium has a pH ranging from 6 to 7.5 for Picochlorum. In some aspects, the growth medium has a pH ranging from 7 to 9 for Ulva. In some aspects, the growth medium has a pH ranging from 9 to 10 for Spirulina. In some aspects, the growth medium has a pH ranging from 2 to 3 for Galdieria.

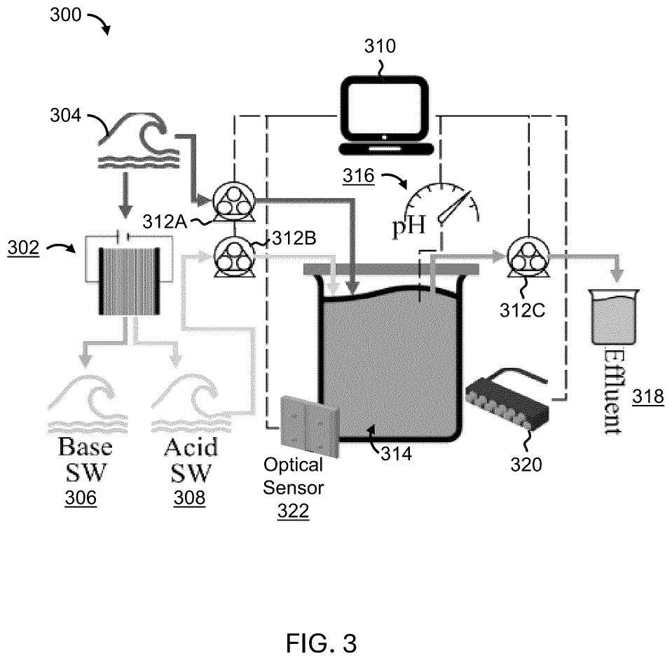

FIG. 3 shows another example of a system 300 for coupling electrochemical marine carbon capture with photosynthesis. Like the system 100 of FIG. 1, the system 300 comprises an electrochemical cell 302 in fluid communication with a saline water source 304. The electrochemical cell 302 converts seawater or brackish water from the saline water source 304 into a base stream 306 and an acid stream 308.

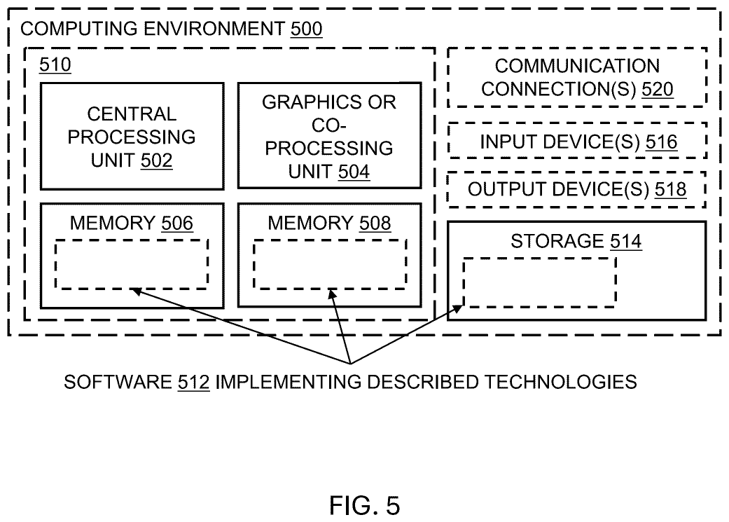

In some aspects, the system 300 further comprises a controller 310. In some aspects, the controller 310 comprises a computing device. Additional aspects of exemplary computing device are described in more detail below with reference to FIG. 5. The controller 310 is communicatively coupled to one or more fluid control devices 312A, 312B, and/or 312C. Any suitable fluid control device can be used. Some examples of suitable fluid control devices include flow meters and dosing pumps.

In the exemplary system 300 of FIG. 3, the fluid control devices 312A and 312B are configured to control a flow rate or add a predetermined amount of a fluid to a growth medium 314 for a photosynthetic organism as specified by the controller 310. For example, the fluid control device 312B can add a predetermined amount of fluid from acid stream 308 to the growth medium 314 and/or maintain a metered flow rate from the acid stream 308 into the growth medium 314. In some aspects, the acid stream 308 can be provided into the growth medium 314 in an amount and/or at a flow rate that delivers 0.1 mM to 5 mM H⁺ ions to the growth medium 314. In some aspects, the acid stream 308 is provided in an amount and/or at a flow rate that delivers 0.8 mM to 2.0 mM H⁺ ions to the growth medium 314.

As introduced above, when added to active cultures of a photosynthetic organism, acidified seawater or brackish water generated by the electrochemical cell 302 alters carbonate-bicarbonate equilibrium within the growth medium 314 and can thereby increase bioavailability of CO₂ and accelerate the photosynthetic organism growth rate. For example, and as described in more detail below, additions of up to 2 mM H⁺ from the acid stream 308 can increase algal productivity up to three-fold. As described in more detail below, high-level analysis conducted based on data suggests that the system 300 can sequester ˜30 kg_{CO2}/kg_{acid} when compared to other means of acid utilization or disposal. Higher net CO₂ removal can be achieved through appropriate choice of photosynthetic organism species, acid addition rates, and other growth conditions (e.g., oxygenation of the growth medium), as will be understood with the benefit of the present disclosure.

In some aspects, the controller 310 is communicatively coupled with a pH sensor 316 that measures a pH of the growth medium 314. In this manner, the controller 310 can monitor the pH of the growth medium 314. Furthermore, in some aspects, the controller 310 can titrate the acid stream 308 to maintain a predetermined pH or pH program in the growth medium 314. For example, the controller 310 can direct the fluid control device 312B to add at least a portion of the acid stream 308 into the growth medium 314, or to reduce an amount of the acid stream 308 being added to the growth medium 314. In some aspects, the controller 310 can use the fluid control device 312C to control a flow rate or remove a predetermined amount of effluent 318 from the growth medium 314. In some aspects, the controller 310 can use the fluid control device 312A to add seawater or brackish water directly from the saline water source to the growth medium 314. This can allow the controller 310 to dilute the growth medium 314.

The controller 310 is, in some aspects, communicatively coupled with one or more additional devices that can control or measure the growth rate of the photosynthetic organism. For example, the system 300 of FIG. 3 includes an energy source 320 (e.g., a grow light, such as an LED). The energy source 320 is configured to illuminate the growth medium 314 and thereby accelerate growth of the photosynthetic organism. The system 300 of FIG. 3 further comprises an optical sensor 322 that can measure one or more optical properties of the growth medium, such as an optical density (OD). The OD can serve as an indicator of the growth of the photosynthetic organism. In this manner, the optical sensor 322 enables the controller 310 to monitor growth of the photosynthetic organism and adjust any suitable parameters accordingly, such as adjusting illumination of the growth medium by the energy source 320 and/or adjusting the chemistry of the growth medium (e.g., by adjusting the pH of the growth medium) in response to the one or more optical properties.

### V. Method

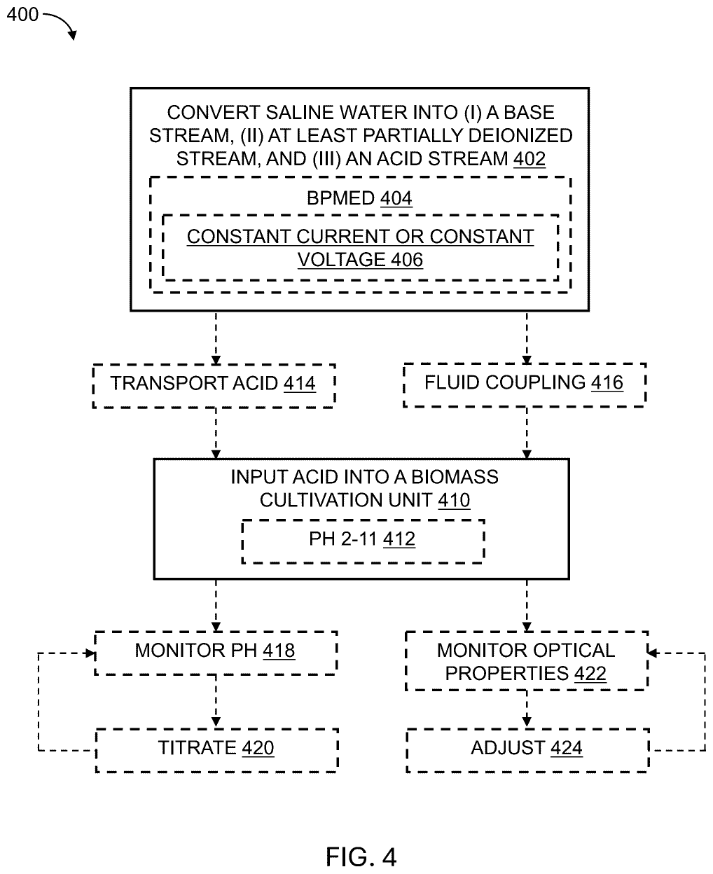

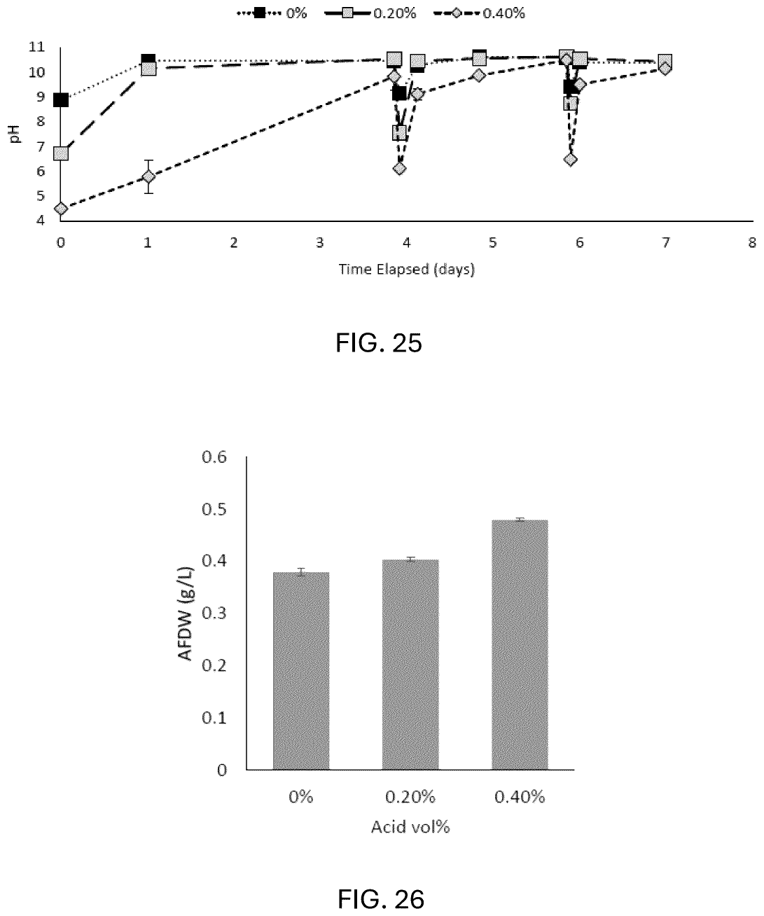

FIG. 4 shows a flow diagram depicting aspects of an exemplary method 400 for coupling electrochemical marine carbon capture with photosynthesis. The following description of the method 400 is provided with reference to FIGS. 1-3 (discussed above) and FIGS. 5-26 (discussed below). It will be appreciated that the method 400 also can be performed in other contexts.

At step 402 of FIG. 4, the method 400 comprises using an electrochemical cell configured to be in fluid communication with a saline water source comprising saline water to convert the saline water into (i) a base stream comprising hydroxide ions, (ii) an at least partially deionized water stream, and (iii) an acid stream comprising hydrogen ions.

As indicated at optional step 404, in some aspects, converting the seawater into the base stream and the acid stream comprises inputting the seawater into a BPMED comprising a plurality of membranes. For example, the BPMED system 200 of FIG. 2 can be used to convert saline water into an acid stream 214 and a base stream 216. In some aspects, and as indicated at optional step 406 of the method 400, the BPMED system is operated in a constant current mode or a constant voltage mode. The current and/or voltage settings of the BPMED system can be selected based upon system size and configuration to meet desired product quality.

At step 410 of FIG. 4, the method 400 comprises inputting the acid stream into a biomass cultivation unit comprising a photosynthetic organism and a growth medium for the photosynthetic organism. In some aspects, and as indicated at optional step 412 of the method 400, the growth medium has a pH ranging from 2 to 11. As described above, the pH can be selected based on the species of photosynthetic organism. In some aspects, the photosynthetic organism comprises a microalgal species, a macroalgal species, a filamentous algal species, a phytoplankton species, a plant species, or a combination thereof. In some aspects, the acid stream catalyzes CO₂ release from the growth medium to accelerate growth of the photosynthetic organism relative to growth without the acid stream and facilitates CO₂ storage by the photosynthetic organism.

In some aspects, and as indicated at optional step 414 of the method 400, contents of the acid stream can be transported to the biomass cultivation unit in one or more batches prior to inputting the acid stream into the biomass cultivation unit. For example, acidified saline water can be transported by the truckload, by railcar, etc. from a saline water electrochemistry plant to the biomass cultivation unit, which may be located offsite.

As indicated at optional step 416 of the method 400, in some aspects, inputting the acid stream into the biomass cultivation unit additionally (or alternatively) comprises fluidly coupling the acid stream with the biomass cultivation unit. For example, an electrochemical cell that produces the acid stream can be co-located with the biomass cultivation unit, or the acid stream can be delivered to the biomass cultivation unit by pipeline. This can reduce carbon emissions associated with transporting the contents of the acid stream between the electrochemical cell and the biomass cultivation unit.

In some aspects, at optional step 418, the method 400 can further comprise monitoring pH of a growth medium for the photosynthetic organism(s). In this manner, and as indicated at optional step 420, the method 400 can include titrating the pH of the growth medium. For example, the controller 310 of FIG. 3 can be used to titrate the pH of the growth medium 314 to maintain a predetermined pH or pH program. This can encourage additional uptake and storage of carbon by the photosynthetic organism.

In some aspects, at optional step 422, the method 400 can further comprise monitoring one or more optical properties of the growth medium. In this manner, and as indicated at optional step 424, the method 400 can include adjusting illumination of the growth medium and/or chemistry of the growth medium in response to the one or more optical properties. Like the pH titration at optional step 420, this can encourage additional growth of the photosynthetic organism relative to not adjusting the illumination and/or the chemistry of the growth medium.

### VI. Computing System

FIG. 5 depicts a generalized example of a suitable computing system 500 in which the described innovations of the present disclosure may be implemented. The computing system 500 is not intended to suggest any limitation as to scope of use or functionality of the present disclosure, as the innovations may be implemented in diverse general-purpose or special-purpose computing systems.

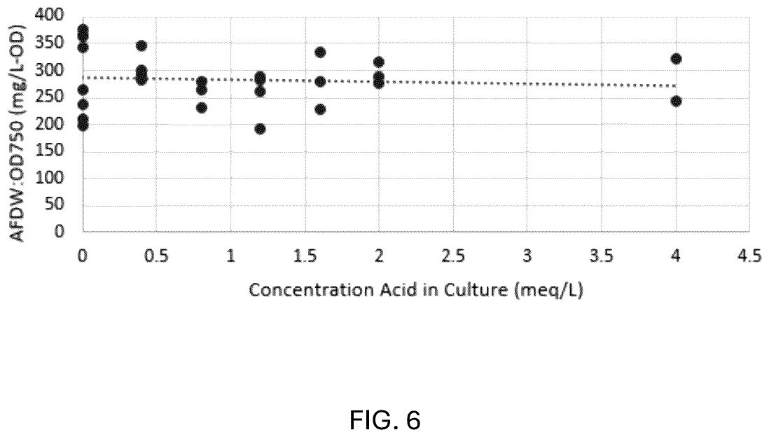

With reference to FIG. 5, the computing system 500 includes one or more processing units (e.g., central processing unit 502 and/or graphics or c-processing unit 504) and corresponding tangible memory 506 and/or 508. In FIG. 5, this basic optional configuration 510 is included within a dashed line. The processing units 502, 504 execute computer-executable instructions, such as for implementing components of the computing environments of, or providing the outputs (e.g., control signals) shown in, FIGS. 1-4 (described above) and FIGS. 6-26 (described below). A processing unit can be a general-purpose central processing unit (CPU), processor in an application-specific integrated circuit (ASIC), or any other type of processor, including any combinations thereof. In a multi-processing system, multiple processing units execute computer-executable instructions to increase processing power. For example, FIG. 5 shows a central processing unit 502 as well as a graphics processing unit or co-processing unit 504. The tangible memory 506, 508 may be volatile memory (e.g., registers, cache, RAM), non-volatile memory (e.g., ROM, EEPROM, flash memory, etc.), or some combination of the two, accessible by the processing unit(s) 502 and/or 504. The memory 506, 508 stores software 512 implementing one or more innovations described herein, in the form of computer-executable instructions suitable for execution by the processing unit(s) 502 and/or 504.

The computing system 500 may have additional features. For example, the computing system 500 includes tangible storage 514, one or more input devices 516, one or more output devices 518, and one or more communication connections 520. An interconnection mechanism (not shown) such as a bus, controller, or network interconnects the components of the computing system 500. Typically, operating system software (not shown) provides an operating environment for other software executing in the computing system 500, and coordinates activities of the components of the computing system 500.

The tangible storage 514 may be removable or non-removable, and includes magnetic disks, magnetic tapes or cassettes, CD-ROMs, DVDs, or any other medium which can be used to store information in a non-transitory way and which can be accessed within the computing system 500. The tangible storage 514 stores instructions for the software 512 implementing one or more innovations described herein.

The input device(s) 516 may be a touch input device such as a keyboard, mouse, pen, or trackball, a voice input device, a scanning device, or another device that provides input to the computing system 500. The output device(s) 518 may be a display, printer, speaker, CD-writer, or another device that provides output from the computing system 500.

The communication connection(s) 520 enables communication over a communication medium to another computing entity. The communication medium conveys information, such as computer-executable instructions, audio or video input or output, or other data in a modulated data signal. A modulated data signal is a signal that has one or more of its characteristics set or changed in such a manner as to encode information in the signal. By way of example, and not limitation, communication media can use an electrical, optical, RF, or other carrier.

The innovations can be described in the general context of computer-executable instructions, such as those included in program modules, being executed in a computing system on a target real or virtual processor. Generally, program modules or components include routines, programs, libraries, objects, classes, components, data structures, etc. that perform particular tasks or implement particular abstract data types. The functionality of the program modules may be combined or split between program modules as desired in various embodiments. Computer-executable instructions for program modules may be executed within a local or distributed computing system. In general, a computing system or computing device can be local or distributed, and can include any combination of special-purpose hardware and/or general-purpose hardware with software implementing the functionality described herein. In various examples described herein, a module (e.g., component or engine) can be “coded” to perform certain operations or provide certain functionality, indicating that computer-executable instructions for the module can be executed to perform such operations, cause such operations to be performed, or to otherwise provide such functionality. Although functionality described with respect to a software component, module, or engine can be carried out as a discrete software unit (e.g., program, function, class method), it need not be implemented as a discrete unit. That is, the functionality can be incorporated into a larger or more general-purpose program, such as one or more lines of code in a larger or general-purpose program.

Described algorithms may be, for example, embodied as software or firmware instructions carried out by a digital computer. For instance, any of the disclosed few-shot machine learning, automation, and montaging techniques can be performed by one or more a computers or other computing hardware that is part of a data acquisition system. The computers can be computer systems comprising one or more processors (processing devices) and tangible, non-transitory computer-readable media (e.g., one or more optical media discs, volatile memory devices (such as DRAM or SRAM), or nonvolatile memory or storage devices (such as hard drives, NVRAM, and solid-state drives (e.g., Flash drives)). The one or more processors can execute computer-executable instructions stored on one or more of the tangible, non-transitory computer-readable media, and thereby perform any of the disclosed techniques. For instance, software for performing any of the disclosed embodiments can be stored on the one or more volatile, non-transitory computer-readable media as computer-executable instructions, which when executed by the one or more processors, cause the one or more processors to perform any of the disclosed techniques or subsets of techniques. The results of the computations can be stored in the one or more tangible, non-transitory computer-readable storage media and/or can also be output to the user, for example, by displaying, on a display device, image segmentations with a graphical user interface.

### VII. Overview of Several Embodiments

Disclosed herein are aspects of a system, comprising: an electrochemical cell configured to be in fluid communication with a saline water source comprising saline water, wherein the electrochemical cell converts the saline water into (i) a base stream comprising hydroxide ions, (ii) an at least partially deionized water stream, and (iii) an acid stream comprising hydrogen ions; and a biomass cultivation unit in fluid communication with the acid stream produced by the electrochemical cell, the biomass cultivation unit comprising a photosynthetic organism and a growth medium for the photosynthetic organism, and wherein the acid stream catalyzes release of CO₂ from the growth medium to accelerate growth of the photosynthetic organism relative to growth without the acid stream and facilitates CO₂ storage by the photosynthetic organism.

In any or all of the above aspects, the electrochemical cell comprises a bipolar membrane electrodialysis system.

In any or all of the above aspects, the bipolar membrane electrodialysis system comprises a plurality of membranes, and wherein the bipolar membrane electrodialysis system is operated in a constant potential mode or a constant current mode.

In any or all of the above aspects, the photosynthetic organism comprises a microalgal species, a macroalgal species, a filamentous algal species, a phytoplankton species, a plant species, or a combination thereof.

In any or all of the above aspects, the photosynthetic organism comprises Picochlorum, Tetraselmis, Chlamydomonas, Tisochrysis, Pavlova, Arthrospira, Spirulina, Galdieria, Limnospira, Cyanobacterium, Asparagopsis, Prionitus, Ulva, Gracilaria, Saccharina, Kappaphycus, Palmeria, Porphyra, Zostera marina, or any combination thereof.

In any or all of the above aspects, the biomass cultivation unit comprises a growth medium for the photosynthetic organism, wherein the growth medium has a pH ranging from 2 to 11 with or without added growth-enhancing nutrients and wherein growth medium is saline water-based.

In any or all of the above aspects, the growth medium has a pH ranging from 2 to 11.

In any or all of the above aspects, the system further comprises: an optical sensor that measures one or more optical properties of the growth medium; and a controller communicatively coupled to the optical sensor, wherein the controller monitors the one or more optical properties of the growth medium and adjusts illumination of the growth medium and/or chemistry of the growth medium in response to the one or more optical properties.

Also disclosed herein are aspects of a method for coupling electrochemical marine carbon capture with photosynthesis, the method comprising: using an electrochemical cell configured to be in fluid communication with a saline water source comprising saline water, wherein the electrochemical cell converts the saline water into (i) a base stream comprising hydroxide ions, (ii) an at least partially deionized water stream, and (iii) an acid stream comprising hydrogen ions; and inputting the acid stream into a biomass cultivation unit comprising a photosynthetic organism and a growth medium for the photosynthetic organism, wherein the acid stream catalyzes CO₂ release from the growth medium to accelerate growth of the photosynthetic organism relative to growth without the acid stream and facilitates CO₂ storage by the photosynthetic organism.

In any or all of the above aspects, the method further comprises transporting contents of the acid stream to the biomass cultivation unit in one or more batches prior to inputting the acid stream into the biomass cultivation unit.

In any or all of the above aspects, inputting the acid stream into the biomass cultivation unit comprises fluidly coupling the acid stream with the biomass cultivation unit.

In any or all of the above aspects, converting the seawater into the base stream and the acid stream comprises inputting the seawater into a bipolar membrane electrodialysis system comprising a plurality of membranes.

In any or all of the above aspects, converting the saline water into the base stream and the acid stream comprises operating the bipolar membrane electrodialysis system in a constant potential mode or a constant current mode.

In any or all of the above aspects, the photosynthetic organism comprises a microalgal species, a macroalgal species, a filamentous algal species, a phytoplankton species, a plant species, or a combination thereof.

In any or all of the above aspects, the growth medium has a pH ranging from 2 to 11.

In any or all of the above aspects, the method further comprises monitoring the pH of the growth medium and titrating the acid stream to maintain a predetermined pH or pH program in the growth medium.

In any or all of the above aspects, the method further comprises monitoring one or more optical properties of the growth medium and adjusting illumination of the growth medium and/or chemistry of the growth medium in response to the one or more optical properties.

Also disclosed herein are aspects of a biomass cultivation unit configured to facilitate CO₂ capture, the biomass cultivation unit comprising: a growth medium; a photosynthetic organism; and an acid stream in fluid communication with the growth medium, the acid stream comprising hydrogen ions derived from electrochemical treatment of saline water, wherein the acid stream catalyzes CO₂ release from the growth medium for use by the photosynthetic organism, and wherein the acid stream accelerates growth of the photosynthetic organism relative to growth without the acid stream.

In any or all of the above aspects, the biomass cultivation unit is in fluid communication with an electrochemical cell configured to be in fluid communication with a saline water source, wherein the electrochemical cell converts saline water from the saline water source into (i) a base stream comprising hydroxide ions, (ii) an at least partially deionized water stream, and (iii) the acid stream.

In any or all of the above aspects, the biomass cultivation unit further comprises a pH sensor that measures a pH of the growth medium; and a controller communicatively coupled to the pH sensor, wherein the controller monitors the pH of the growth medium and titrates the acid stream to maintain a predetermined pH or pH program in the growth medium.

### VIII. Examples

Aspects of the present teachings can be further understood in light of the following examples.

### Example 1—BPMED Acid Generation

A bench-scale BPMED unit purchased from PCCell GmbH (ED 64004-T10-1631-ED1, Heusweiler, Germany) was used to generate acid and base streams using filtered natural seawater obtained from Sequim Bay, WA (obtained using 40 μm filters from AMIAD WATER SYSTEMS®, Mooresville, NC). The unit comprised five cell pairs, with each composed of cation, bipolar, and anion membranes placed in an order and orientation specified by the manufacturer. The membranes were placed between a Pt/Ir-MMO coated Ti anode and a stainless-steel cathode. A sodium sulfate rinse (0.25M, ACS grade >99.0%, SIGMA-ALDRICH®, St. Louis, MO) was circulated through the electrode chambers to prevent secondary reactions. Peristaltic pumps (MASTERFLEX® 77410-10, MASTERFLEX® 7528-10, MITYFLEX® 400-205-90127-1, AV AVANTOR®, Radnor, PA) were used for all streams through the BPMED and set to flow rates between 400-1200 mL/min. A KEITHLEY® 2230-30-6 power supply (TEKTRONIX®, Beaverton, OR) was used for powering the BPMED and associated software (KICKSTART®) to record current/voltage data. Evaluations were conducted in constant current mode with current densities ranging from 1.25 to 10 mA/cm², which corresponded to full-device voltages between 6.5 and 15.0 V and was under the manufacturer recommended maximum of 3 V per membrane. The pH of each stream was monitored with a lab-grade pH probe (ENV-40-pH, Atlas Scientific, Long Island City, NY), directly inline after the BPMED unit. The probes were polled and calibrated by an ARDUINO® UNOR (ARDUINO®, Italy); custom code in the supplied IDE was used to log data to a text file at 0.2 Hz. The acid stream generated from the BPMED was stored in closed, chemical-safe containers until used for algae growth. The base and de-ionized seawater streams were disposed following standard laboratory safety procedures.

Energy consumption by BPMED can be modeled using the following equation:

$$Energy consumption = ( \frac{i * {E}_{avg} * {t}_{on}}{{v}_{total} * ( {10}^{- pH} final - {10}^{- pH} initial )} ) \qquad ( 1 )$$

$$Q = 400 mL / min$$

In Equation (1), i=current in amps, E_{avg}=average voltage, tₒₙ=time operating in hours, vₜₒₜₐₗ=sample volume in liters (˜4 L), and pHᵢₙᵢₜᵢₐₗ and pH_{final} are the measured pH values at start time and 30 minutes into the run. Q=volumetric flow rate; as noted, this was evaluated at a constant flow rate of 400 mL/min.

### Example 2—Biomass Growth and Monitoring

Picochlorum celeri, a fast-growing, light-tolerant marine alga was used as a model organism for measuring increased algal growth rates in BPMED acidified seawater. Algal well plate testing evaluations were conducted in 24-well plates and sealed with transparent, self-adhesive covers. Initial and final chlorophyll-A fluorescence levels were measured at 490 nm excitation and 680 nm emission wavelengths to test for activity in photosystem II using an automated plate reader (BIOTEK CYTATION® 3). pH was measured immediately after acid addition and again after 72 hrs when the plate seal was broken to allow for the insertion of the pH electrode.

Growth evaluations were conducted in 250-mL Erlenmeyer flasks illuminated by a multi-spectrum LED panel (VS3000 LED Grow Light, VIVOSUN®, Ontario, CA). The panel provided incident light intensity of approximately 600 μmol/m²·s as measured using a LI-COR® LI-190R quantum sensor (LI-COR®, Lincoln, NE, USA) placed within the Erlenmeyer flask at the surface of the growth medium. Temperatures within the flask system were approximately 27.3±1.5° C. during the daylight hours and 20.2±0.9° C. during the night. Cultures were grown in 200-mL volumes of filtered natural seawater from Sequim Bay supplemented with f/2 nutrients with 0.88 mM N and 0.036 mM P. Duplicate flasks were set on magnetic stir plates and mixed at 130 RPM with 4-cm magnetic stir bars. Flasks were topped with porous foam stoppers and semi-micro pH probes were fed through the sides of the stoppers (ACCUMET® 13-620-290, Fisher Scientific, USA). A programmable aquarium controller system (Apex, NEPTUNE SYSTEMS®, Morgan Hill, CA) was used to monitor pH and temperature at 10-minute increments. The system was also used to control lights on a 12 hr:12 hr photoperiod, perform acid addition via peristaltic pumps, and control air sparging via gas solenoid valves.

Mother cultures used as exemplary inocula were acclimated in the exemplary growth medium at least 1 week prior to the evaluation with constant air sparging under 500 μmol/m²·s light on a 12 hr:12 hr cycle. Culture optical densities at 750 nm and 680 nm (OD₇₅₀ and OD₆₈₀, respectively) were measured, with initial measurements taking place prior to testing cultures. The ratio of the two (OD 680/750) was used indicate relative chlorophyll-A content and overall culture health. Evaluations were started when mother culture OD_{680/750} was consistent (greater than 1.4). Mother culture density was held by periodically diluting to an OD₇₅₀ value between ca. 0.3-0.4 to keep the culture in an active linear growth state.

Initial acid-enhanced algae growth were performed over a 24-hour period. Algae were inoculated to a starting optical density of 0.1 OD₇₅₀. Daily measurements of optical density (OD₆₈₀ and OD₇₅₀) along with alkalinity were taken approximately once per 24 hours, using a dual-beam spectrophotometer (GENESYS® 10UV-Vis, THERMO SCIENTIFIC®). Cultures were centrifuged to remove cells and alkalinity was measured by titration of 30-mL supernatant with 5 mN H₂SO₄.

Due to the relatively small amount of total biomass generated in the photobioreactors shown here, an additional metric became necessary to quantify growth. Cell biomass at the end of the evaluations was measured as AFDW by using an average AFDW per unit optical density conversion. Briefly, culture samples were filtered onto pre-ashed and tared 0.7 μm glass fiber filters (GF/F WHATMAN®, CYTIVA® Life Sciences, Marlborough, MA). Filters with algal biomass were dried overnight at 105° C., cooled in a desiccator and weighed. After determining the dry weight, samples were ashed for 2 hours at 540° C. to remove volatile matter. AFDW was calculated as the mass difference between dried and ashed algae samples per volume culture.

A series of 28 tests were run in which single acid addition was performed at different quantities (meq/L), including inoculated cultures prior to acid addition. In each case, samples for AFDW calculation were extracted starting with three different volumes: 10, 20, and 30 mL. Additionally, OD₇₅₀ values were taken for each exemplary culture volume. AFDW values were correlated to OD₇₅₀ measurements to determine approximate biomass density in mg/L. FIG. 6 shows a plot of correlation between measured AFDW:OD₇₅₀ ratio across multiple acid addition tests. No distinct trend was observed for the AFDW:OD₇₅₀ correlation as a function of acid added, giving confidence to the application of the correlation over the conditions tested. An average correlative value of 283±9 mg AFDW/L-day (standard error 9 mg/L-day, n=28) was determined and was used to convert optical density measurements to quantify gravimetric mass over time.

### Example 3—Characterization of BPMED-Generated Acid

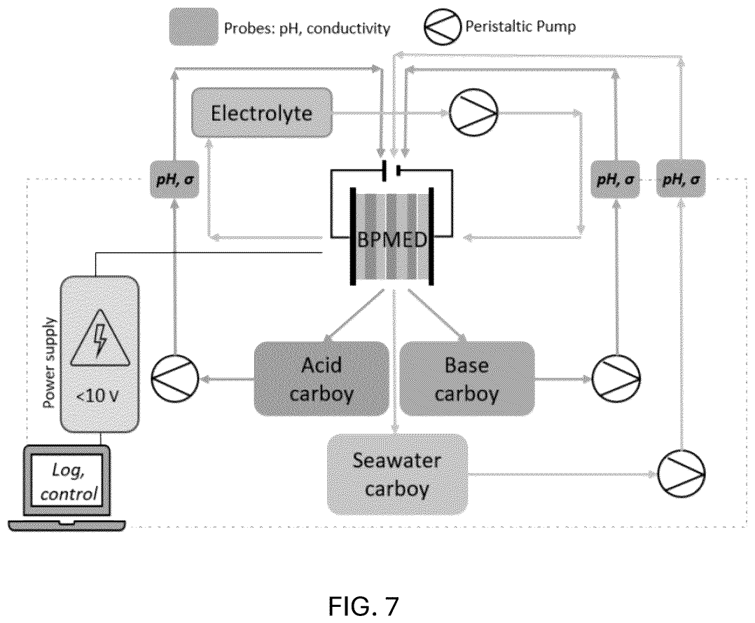

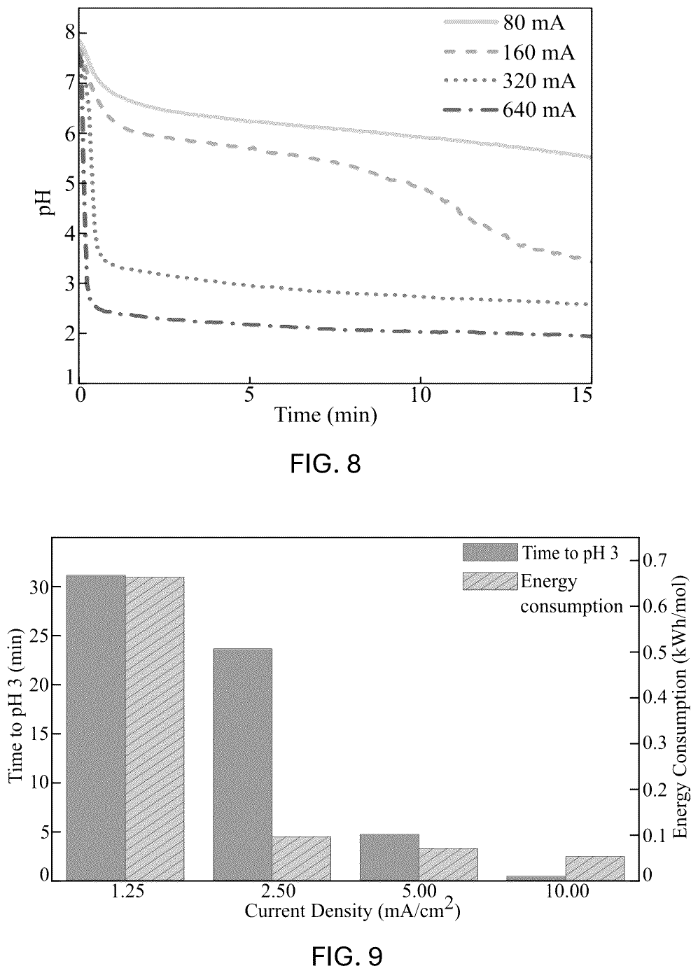

FIG. 7 shows a detailed process flow diagram describing the operational configuration of a BPMED unit. As described above with reference to FIG. 2, the BPMED unit comprises a stack including an AEM, CEM, and BPM alternating with thin polymer separators placed between an anode and a cathode. When filtered seawater is flowed through the system and a potential is applied across the stack, water splitting reactions at the surface of the BPM generates H⁺ and OH⁻ ions, and the placement of the AEM and CEM directs the electromigration of the anions and cations to generate alternating streams of acidic, basic, and deionized streams. To evaluate the acid pH as a function of operational parameters, the BPMED unit was operated under varying constant current values (FIG. 8) and the resulting pH of the acid stream was monitored over time. Higher current densities yielded higher initial pH gain rates and more extreme saturation pH values. With BPMED effluents being recirculated, the time to generate acid of pH 3 as a function of varying current densities at constant flow rate was measured (FIG. 9). The non-linearities present in the production rates were expected, given space charge restrictions at low cross-membrane potentials, kinetic limitations at higher cross-membrane potentials, and electro-osmotic flow back across membranes at high acid/base concentrations. In each case, the energy consumed per mole of acid generated was calculated (FIG. 9) as kWh used (current*average voltage*on-time) per mol of H⁺ generated (sample volume in 30 min*[10^{-pH_final}−10^{-pH_initial}], at an average flow rate of 400 mL/min). Higher current densities correlated with increased energy efficiencies as the system reached pH 3 faster than at lower current densities. The acid generated using the BPMED system was then stored in sealed containers to prevent air-seawater CO₂ exchange until it was used for algae growth evaluations.

### Example 4—Acid Use for Enhanced Algae Growth

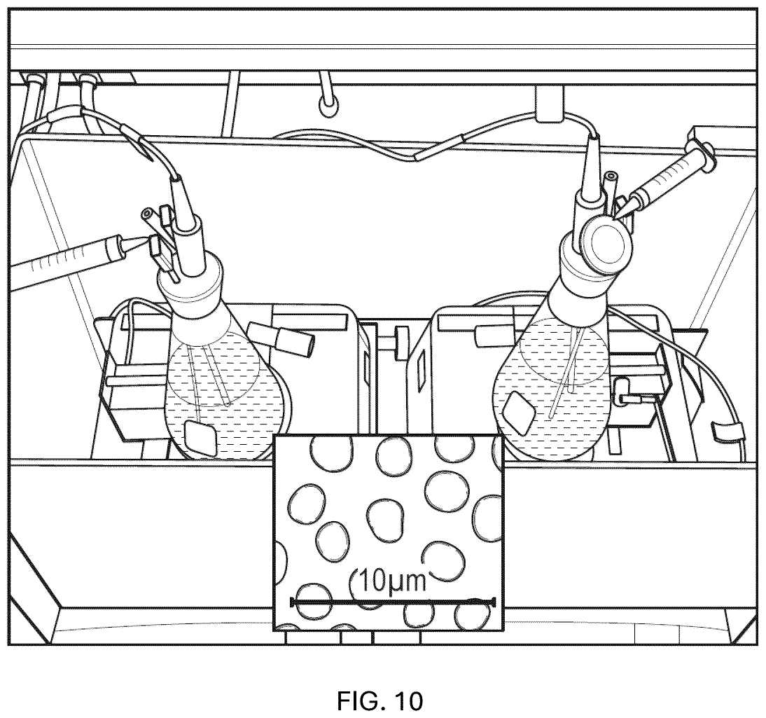

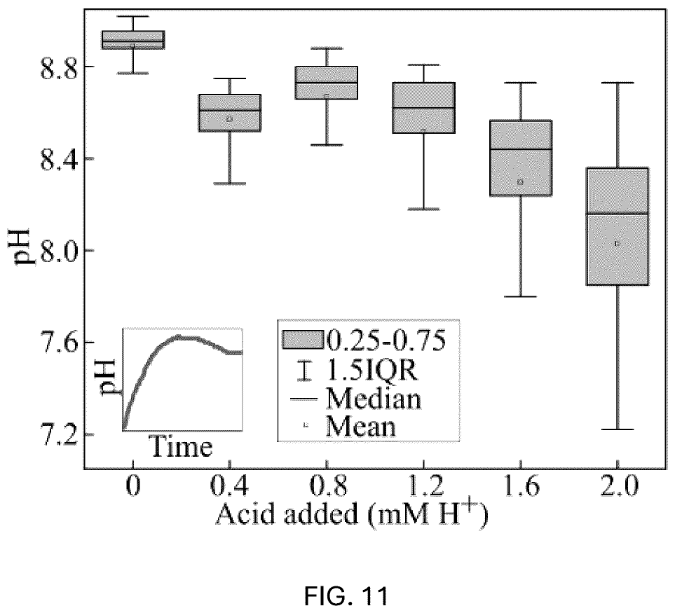

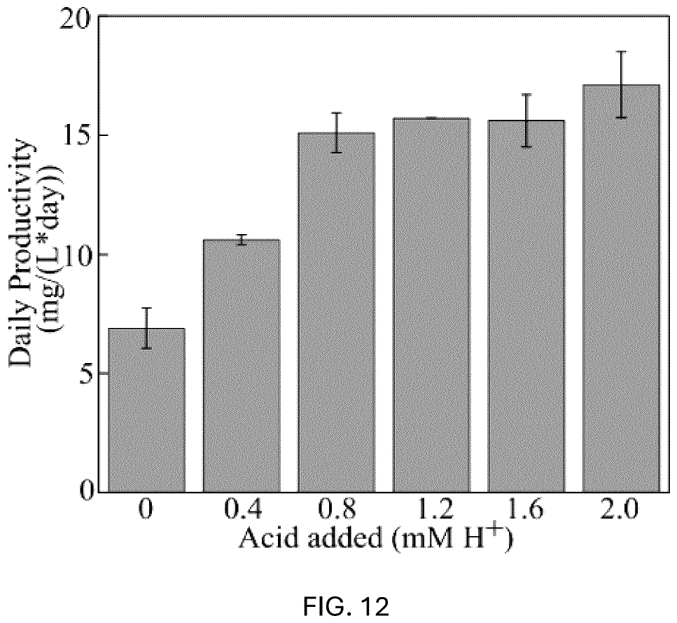

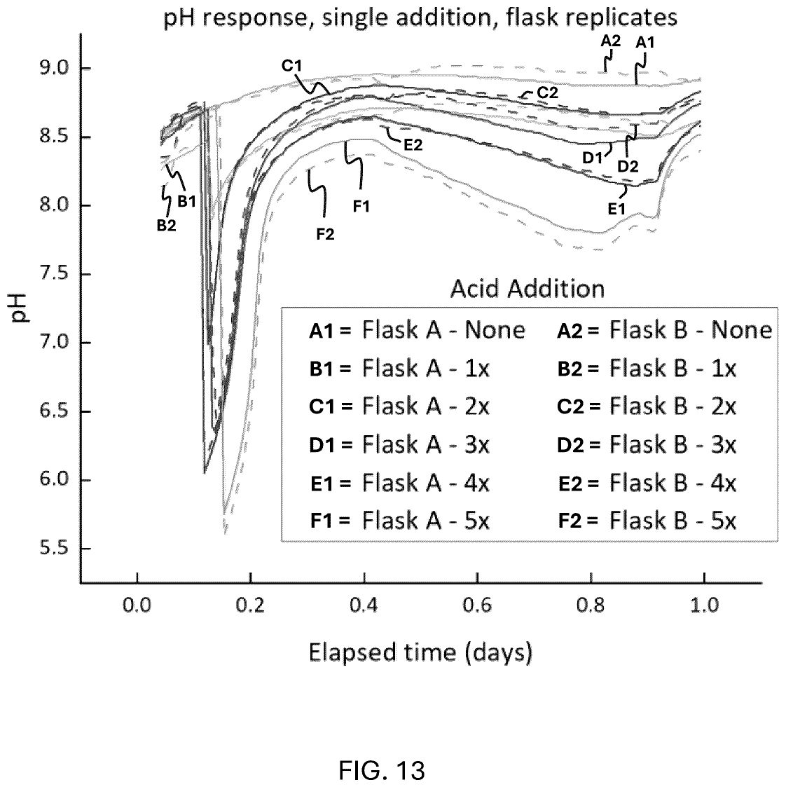

A schematic representation of the setup used to grow P. celeri with BPMED acid is described above with reference to FIG. 3. Given difference in rates of acid production by BPMED vs. acid consumption for algal cultivation used in this study, the acid was stored in a container and not directly pumped into the growth chamber. FIG. 3 is a simplified representation of the process when the rates of acid production and consumption match. An illustration of bench-scale growth chambers used in the study is provided as a reference in FIG. 10. The pH of the growth media in the PBR was monitored using a computer-controlled system and peristaltic pumps were used for influent and effluent flow from the chamber, and acid was added to the chamber at pre-determined intervals and in pre-determined volumes. The changes to culture pH increased with increase in the volumes of acid added (FIG. 11). In general, the culture pH tended to dip rapidly upon acid addition but rise with algal CO₂ uptake to reach eventual settling (FIG. 11, inset). These evaluations demonstrate the feasibility to use acids additions to control the pH within biologically relevant pH regimes in the actively growing culture. When the BPMED acid is added to the seawater-based medium it shifts the naturally present carbonate equilibrium from bicarbonate dominant towards carbonic acid, which results in gaseous CO₂. CO₂ (g) can readily diffuse across the cell membrane without the need for active transport. Once inside the cell, CO₂ is transferred via a series of carbonic anhydrase enzymes to the carbon fixation centers where it contributes to photosynthate production via RuBisCO. Biomass production is generally limited by the RuBisCO carbon fixation step. High productivity microalgae grown for biofuel production have been shown to be significantly more productive if CO₂ is readily available. The cost of introducing CO₂ into the algal cultivation systems for enhanced productivity is significant and remains one of the largest drivers of the minimum biomass selling price, a key parameter in determining the economic viability. This demonstrates the feasibility of sourcing CO₂ via acid addition to seawater. A clear increase in algal productivity was observed upon acid addition (FIG. 12), with maximum gains observed when 0.8 mM H⁺ was added and 2 mM H⁺ addition yielding roughly 3× the daily growth productivity of non-acidified culture medium. Data show diminishing returns at higher acidic addition levels (lower pH values). Without wishing to be bound by theory, this may be due to the rapid CO₂ release from seawater upon acid addition compared to algal uptake. One approach to increasing CO₂ uptake by the algae (and reducing its loss to the atmosphere) is dosing smaller quantities of acid per addition and having multiple additions over the evaluation period vs. a single large acid addition. Preliminary evaluations with such multiple additions to introduce the same acid quantity as a single addition show increased algal CO₂ uptake (FIG. 13). The optimum growth pH is species-specific and the presumed ideal growth pH for P. celeri is between pH 6.5 and ca. 7.0.

Using a BIOTEK CYTATION® imaging well-plate scanner, chlorophyll fluorescence in the different BPMED acid treatments, which corresponds to photosynthetically active biomass, was measured after 72 hours. Picochlorum tolerated 10% acid addition extremely well, whereas a sharp drop-off at 20% acid was noted.

TABLE 1
Acid addition to algae growth evaluations.

| | % BPMED Acid | | | | | |
|---|---|---|---|---|---|---|
| | 0% Control | 0.01% | 0.1% | 1% | 10% | 20% |
| Initial pH | 8.06 | 7.62 | 7.62 | 7.61 | 3.6 | 2.59 |
| Final pH | 8.96 | 8.86 | 8.92 | 8.88 | 8.29 | 3.30 |

TABLE 2
Acid addition volumes and concentrations used in growth flask
evaluations. The pH of the BPMED acid was 1.64 and the growth
flask volume that the acid was added to was 200 mL.

| | Volume of acid | Acid concentration | H⁺_{eq} | % v acid |
|---|---|---|---|---|
| Sample | added (mL) | (mM) | (μM) | added |
| Control | 0 | 0 | 0 | 0% |
| 1x acid | 2.8 | 0.316 | 316 | 1.4% |
| 2x acid | 5.6 | 0.624 | 624 | 2.7% |
| 3x acid | 8.4 | 0.923 | 923 | 4.0% |
| 4x acid | 11.2 | 1.215 | 1215 | 5.3% |
| 5x acid | 14 | 1.499 | 1499 | 6.5% |

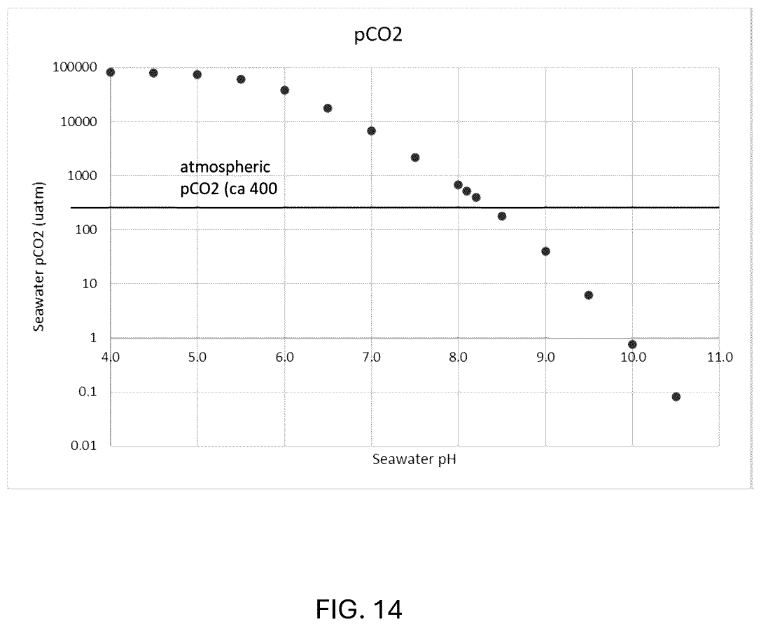

FIG. 14 shows a plot of a calculated relationship between seawater pCO₂ and seawater pH. Atmospheric pCO₂ is indicated by a solid line for reference.

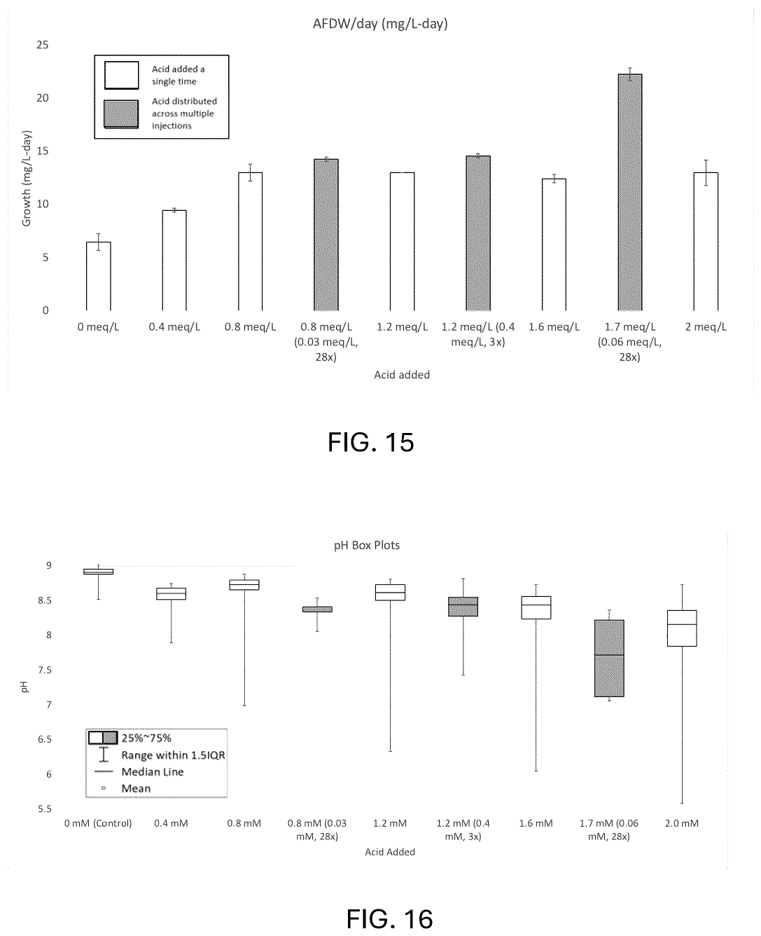

FIGS. 15 and 16 also demonstrate increased growth rates by acid metering. Three additional evaluations were carried out, wherein acid addition was distributed across multiple injection events, equally dispersed across the evaluation time period. FIG. 15 shows a comparison of algal growth between single and multiple acid additions; note 1.7 meq/L addition was peristaltic pump overshoot from a 1.6 meq/L target. There appear to be gains from multiple acid addition to deliver the same total dose, outside the bounds of standard error. FIG. 16 shows quartile box plots of culture pH over the course of the evaluation window.

The evaluations herein show that the pH of the growth medium may be above the ideal growth window for this organism, highlighting the potential to achieve further increase in growth rates upon additional optimization. Some variables that can be considered include the air-seawater CO₂ equilibrium and the buffering capacity of the seawater (alkalinity), in addition to growth pH for the specific species to determine suitable acid addition schemes.

### Example 5—Greenhouse Gas (GHG) Emissions Estimation

The following paragraphs describe aspects of a model of a commercial process used as a baseline for identifying material and energy flows within and through the process boundary. The end use of the acid solution is the focus of the carbon emissions analysis, so seawater pre-treatment, and the acid production in the BPMED device were not considered for simplicity. There are two potential processes considered. In the first scenario, the produced acid is transported by truck to a distributor. It is assumed that the acid solution has a direct end-use without additional treatments; possible uses may include metals corrosion control for industrial use and packaging, sugar extraction for food production, or resin regeneration for water treatment. The distributor is assumed to be 100 miles away with dedicated trucks returning empty to the production site from the distributor. For the second scenario, the dilute acid stream is diverted to a co-located algae cultivation process to promote growth, boost photosynthetic carbon uptake, and generate biomass. The algal biomass is harvested and dewatered. The extent of dewatering can vary, resulting in slurry concentrations of at least 20% algae solids. The dewatered algae have several possible destinations, including biofuel production, ocean carbon sequestration (deep sea transportation and sedimentation), or food and product manufacturing, which have the potential to capture carbon and/or reduce its production. Regardless of the sequestration scenario, this model assumes that carbon is stored for a 100-year timeframe, and analysis ends where the algae is delivered. The life-cycle inventory for algae cultivation includes inputs of carbon dioxide and supplemental nutrients (e.g., nitrogen, phosphorus, and trace metals). The removed water is recycled to the cultivation pond.

The aforementioned process description informed the life-cycle inventory, which was then used to estimate the greenhouse gas emissions for selected scenarios. For the estimation of greenhouse gas emissions, a functional unit was defined as 1 kg of acid solution. The GREET model developed by Argonne National Laboratory was used to calculate the greenhouse gas emissions of the described scenarios; inventory databases within the GREET tool were used. A 2023 update of the GREET database was used.

For the transportation of the dilute acid solution and the algal slurry, it was assumed that the 100 mi. travel distances used heavy-duty trucks fueled by diesel. Dedicated vehicles return to the production site when emptied. The resulting emissions value is 0.0122 kg CO₂/kg of transported material. Normalized transportation for a 3 wt. % solution of acid is 0.41 kg CO₂/kg HCl.

An existing modeled process within GREET, titled “Algae Growth and Dewatering for CAP (2021 SOT)” was used as a baseline for creating a new process specific to the proposed cultivation process described in this work. The selected GREET process is relevant because data for the cultivation of P. celeri was incorporated into the GREET database, which is the utilized species in the evaluations described herein. The material and energy flows to produce one functional unit of algae biomass were assumed to be unchanged with the exception of CO₂. CO₂ was assumed to be air-captured and was thus specified as such in the modified GREET process, whereas template models use anthropogenic CO₂ sources. Electrical energy, nitrogen and phosphorus nutrients, and construction materials for the algae ponds are included in the life-cycle inventory for algae cultivation. The resulting estimate for net GHG emissions, excluding transportation, is −1.9051 kg CO₂/kg of 20 wt. % algal slurry. Acid dosing of 0.8 mM H⁺ is selected as the concentration of solution used to boost cultivation. From the data presented in FIG. 12, it is estimated that 0.64 kg of algae are produced from the addition of 1 kg of HCl. Therefore, −6.1 kg CO₂/kg HCl is sequestered in the algae biomass in this process pathway.

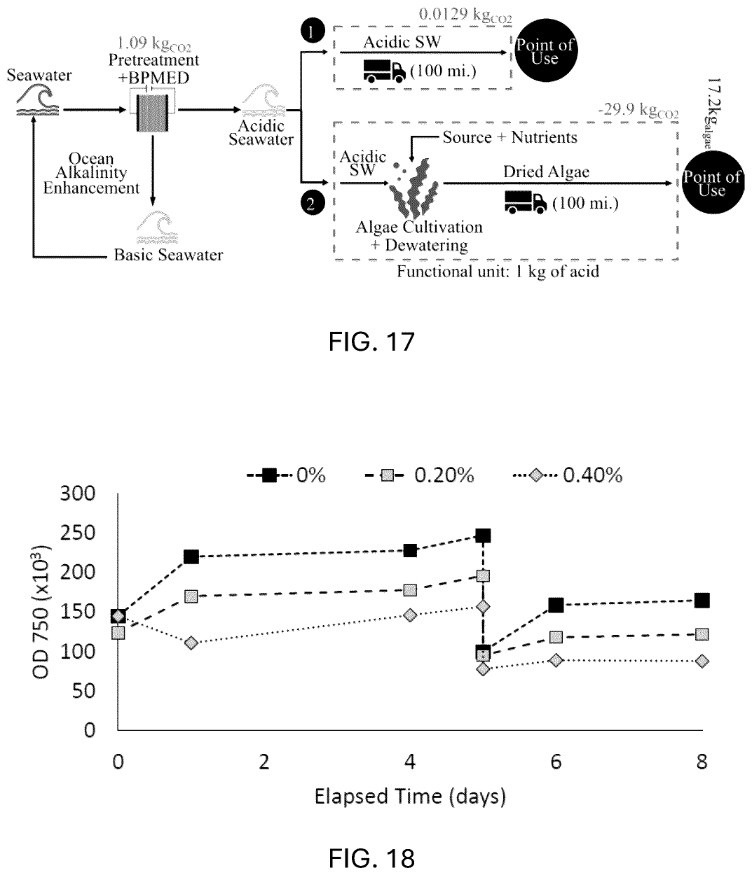

For a functional unit of 1 kg of acidified seawater effluent, the added process of algal cultivation provides a net reduction of CO₂; whereas transport of acid effluent accounts for emission of +0.0129 kg_{CO2}, algal cultivation and dewatering results in sequestration of −29.9 kg_{CO2} (FIG. 17). The 0.0129 kg_{CO2} generated via transport in FIG. 17 is more than suppressed by the retention of 29.9 kg_{CO2} via BPMED effluent acid-enhanced algae growth.

### Example 6—Acid Tolerance of Algal Species

Transitioning from bench-scale to demonstration-scale BPMED (a factor of 10× to 100× growth) resulted in differences in pH of the acid stream. The demonstration unit using a closed loop process generated a more concentrated acid stream (pH 0.79) compared to the bench-scale device (pH 1.68) that used a single-pass configuration. The difference in the pH directly translates to the amount of acid that is used to achieve the same photosynthetic productivity enhancement in the algae—a smaller volume of more concentrated acid vs. larger volumes of less concentrated acid is used to generate the same outcomes.

To account for these differences, a survey of the acid tolerance for various algal species was conducted using the more concentrated acid generated by the demonstration unit operating on Sequim Bay seawater. The acid tolerance was tested on select macroalgae and microalgae cultures using a multi-well plate assay measuring the relative fluorescence intensity of chlorophyll after 24 h (Tables 3-5). The pH of the wells increased and were basic in cases where the cultures survived and were photosynthetically active, consuming the CO₂ in the media. The pH of the wells where the cultures did not survive remained acidic. Note that the evaluations generally showed tolerance <1 vol. % acid and all species were active up to 0.4 vol. % acid. It is possible that they are tolerant of higher acid vol. %, between the step change of 0.4 vol. % and 1 vol. % tested.

TABLE 3
Acid tolerance of Ulva expansa to acid generated
by demonstration-scale BPMED at PNNL-Sequim in sealed
multi-well plates; n = 4 for each tested condition.

| | Acid Concentration (vol. %) | | | | | |
|---|---|---|---|---|---|---|
| | 0 | 0.1 | 0.2 | 0.4 | 1 | 2 |
| Row | Relative Chlorophyll Fluorescence (120 hours) | | | | | |
| A | 17822 | 9404 | 18829 | 10172 | 1858 | 70 |
| B | 16703 | 7846 | 10228 | 25128 | 1622 | 1400 |
| C | 38500 | 20595 | 8829 | 12567 | 1892 | 969 |
| D | 27381 | 5487 | 13617 | 16759 | 1300 | 2974 |

TABLE 4
Acid tolerance of Sequim Bay phytoplankton to acid generated
by demonstration-scale BPMED at PNNL-Sequim in sealed
multi-well plates; n = 4 for each tested condition.

| | Acid Concentration (vol. %) | | | | | |
|---|---|---|---|---|---|---|
| | 0 | 0.1 | 0.2 | 0.4 | 1 | 2 |
| Row | Relative Chlorophyll Fluorescence (24 hours) | | | | | |
| A | 22315 | 24891 | 25733 | 24983 | 28242 | 10832 |
| B | 26362 | 25684 | 24170 | 24534 | 25940 | 11649 |
| C | 22005 | 25228 | 25569 | 25111 | 25654 | 11775 |
| D | 26320 | 27057 | 29587 | 23035 | 25354 | 11614 |

TABLE 5
Acid tolerance of Tetraselmis suecica UTEX2286 to acid
generated by demonstration-scale BPMED at PNNL-Sequim in
sealed multi-well plates; n = 4 for each tested condition.

| | Acid Concentration (vol. %) | | | | | |
|---|---|---|---|---|---|---|
| | 0 | 0.1 | 0.2 | 0.4 | 1 | 2 |
| Row | Relative Chlorophyll Fluorescence (24 hours) | | | | | |
| A | 16252 | 18283 | 18774 | 22016 | 245 | 216 |
| B | 15260 | 16654 | 18889 | 20238 | 273 | 188 |
| C | 13951 | 16216 | 16060 | 18984 | 338 | 229 |
| D | 13441 | 15923 | 16209 | 21425 | 276 | 207 |

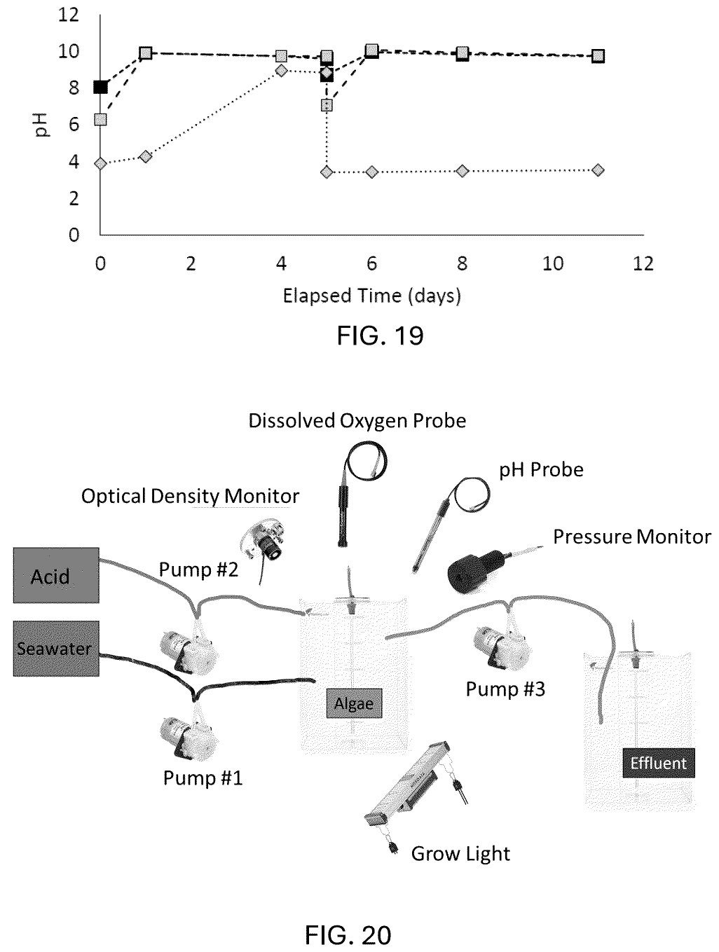

The acid tolerance tests suggest the highest-possible acidic pH for survival but does not determine the rate or frequency of addition of acid that is used to grow the photosynthetic species. To test algal growth with acid addition, evaluations were conducted in larger (100 mL) volume flasks. 100-mL flask cultures of T. suecica UTEX2286 were created based on the results of the well-plate surveys. Acid was added to algal cultures in a single addition at 0 vol. %, 0.2 vol. %, or 0.4 vol. % in enriched Sequim Bay seawater (PROLINE® F/2, PENTAIR AQUATIC ECO-SYSTEMS®). Cultures were incubated in over 8 days while tracking both pH and OD₇₅₀ which was used as a proxy for biomass density (FIGS. 18-19). At day 5, the cultures were diluted by half and additional acid was added.

Growth and change in pH for the 0 vol. % and 0.2 vol. % acid cultures occurred primarily within the first 24 hours of being sealed. The 0.4 vol. % acid culture lagged and showed an initial decrease in OD₇₅₀, this could be due to inhibition from the acidic environment and differences in translating evaluations from the multi-well plate to flask set ups. This is seen with the second addition of acid, which brought the initial pH of the 0.4 vol. % acid culture below pH 3.5, which was fatal to the algae.

These evaluations in sealed flasks indicated that algal culture growth was stifled (note the rapid rise of both pH and OD followed by a plateau of no change). Several factors could be limiting growth. Likely inhibitors of microbial growth are usually either a lack of nutrients (e.g., N, P, C) or the buildup of metabolic waste products (e.g., oxygen). One way to eliminate these growth constraints is to shift to a continuous or semi-continuous flow of enriched seawater into the reactor. This flow of new medium provides the algal cultures the carbon and nutrients for growth and the acid addition makes the bicarbonate in seawater more available by turning it into CO₂. Continuous flow would also reduce excess oxygen within the reactor. An example of a continuous-flow bioreactor design is depicted in FIG. 20.

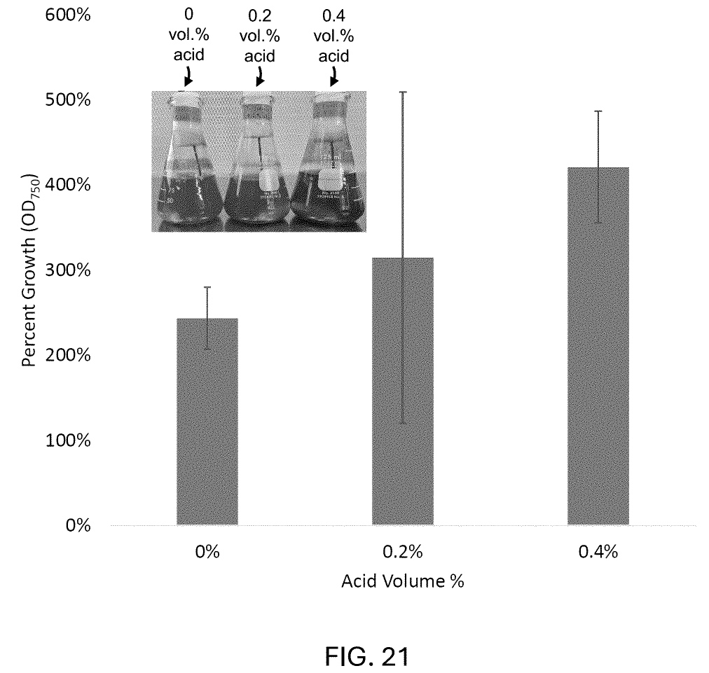

Growth within the sealed flasks was suspected to be limited by a lack of total carbon (CO₂, bicarbonate) in the media, and possibly nitrogen and phosphorous. The growth medium was changed to PROLINE® F/2 with 10× nitrogen and phosphorous content (8.8 mM N and 0.36 mM P) and, after incubating cultures for several days in sealed flasks with acid addition, the cultures were then sparged with air for four days (FIG. 21) to eliminate oxygen build-up and introduce additional atmospheric CO₂.

The 0.4 vol. % acid culture grew the most from air sparging and appeared greener and healthier than the other two culture conditions. The increased growth rate in response to air sparging after acid incubation prompted the inclusion of a sparging step to prevent O₂ inhibition and CO₂ limitation following the acid addition and sealed incubation.

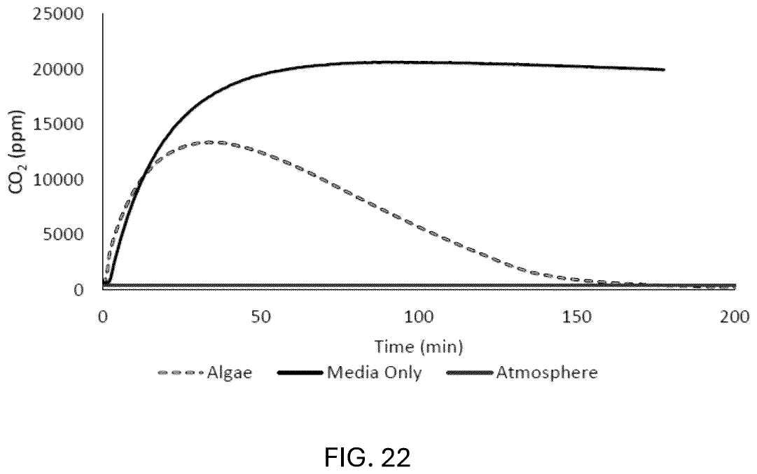

To determine the timing for switching between sealed incubation and air sparging, the CO₂ in the headspace of a sealed flask was measured over time after the addition of 0.4 vol. % acid to either an algae culture or an abiotic growth medium (FIG. 22).

The CO₂ concentration increased to 30-50 times that of the atmosphere in the flask headspace upon acid addition. In the flask containing algae, the CO₂ concentration was observed to decrease to atmospheric levels over approximately 3 hours as the algae consumed the CO₂ for photosynthesis. In the flask without the algae, the CO₂ levels remained high with little observed decrease.

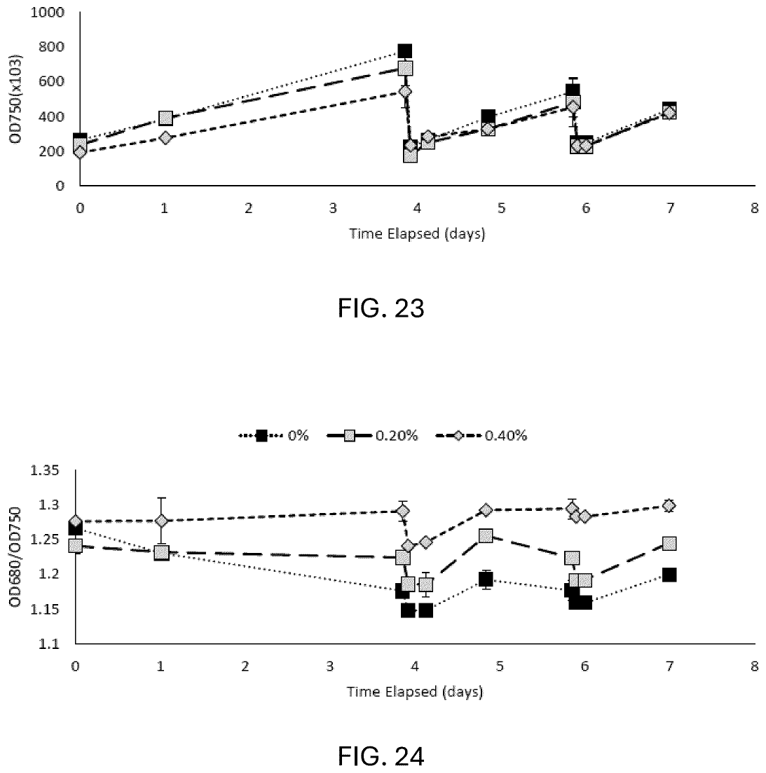

A growth cycle was designed combining both acid addition and air sparging; after creating or diluting an algae culture with fresh media, acid was added to release bicarbonate/bicarbonate, i.e., dissolved inorganic carbon (DIC) as CO₂ and the culture was sealed for 3 h before being air sparged. After the optical density of the culture had approximately doubled, the culture was diluted to restart the cycle. The pH and optical densities of the cultures were tracked for several dilution cycles (FIGS. 23-25).

The change in OD₇₅₀, which indicates the presence of large particles like alga cells, was not notably different between the 0 vol. %, 0.2 vol. %, and 0.4 vol. % acid cultures. The acidified cultures still showed a darker green color than the culture without acid. Additionally, the ratio of OD₆₈₀, which is more indicative of light absorbance by chlorophyll, to OD₇₅₀ was consistently higher for acidified cultures than for the culture without acid, suggesting better algal health.

The AFDW was measured for each culture to determine the concentration of algae more accurately by first drying a sample to determine its solids content, then combusting the solids to determine the fraction of organic compounds (FIG. 26). The samples were taken on day 7 where all three culture conditions had similar OD₇₅₀ readings (FIG. 23). The 0.4% acid culture had 25% higher AFDW than the control culture (0% acid addition) despite similar optical density readings, indicating that more biomass was fixed as carbon in the acid added cultures.

While preferred embodiments of the present disclosure have been shown and described herein, it will be apparent to a person of ordinary skill in the art that such embodiments are provided by way of example only. Variations, changes, and substitutions to these disclosed embodiments will be apparent to a person of ordinary skill in the art without departing from the present disclosure. It should be understood that all such various alternatives to the embodiments described herein may be employed in practicing the present disclosure. The following claims define the scope of the disclosure.

We claim:

1. A system, comprising:
an electrochemical cell configured to be in fluid communication with a saline water source comprising saline water, wherein the electrochemical cell converts the saline water into (i) a base stream comprising hydroxide ions, (ii) an at least partially deionized water stream, and (iii) an acid stream comprising hydrogen ions; and
a biomass cultivation unit in fluid communication with the acid stream produced by the electrochemical cell, the biomass cultivation unit comprising a photosynthetic organism and a growth medium for the photosynthetic organism, and wherein the acid stream catalyzes release of CO₂ from the growth medium to accelerate growth of the photosynthetic organism relative to growth without the acid stream and facilitates CO₂ storage by the photosynthetic organism.

2. The system of claim 1, wherein the electrochemical cell comprises a bipolar membrane electrodialysis system.

3. The system of claim 2, wherein the bipolar membrane electrodialysis system comprises a plurality of membranes, and wherein the bipolar membrane electrodialysis system is operated in a constant potential mode or a constant current mode.

4. The system of claim 1, wherein the photosynthetic organism comprises a microalgal species, a macroalgal species, a filamentous algal species, a phytoplankton species, a plant species, or a combination thereof.

5. The system of claim 1, wherein the photosynthetic organism comprises Picochlorum, Tetraselmis, Chlamydomonas, Tisochrysis, Pavlova, Arthrospira, Spirulina, Galdieria, Limnospira, Cyanobacterium, Asparagopsis, Prionitus, Ulva, Gracilaria, Saccharina, Kappaphycus, Palmeria, Porphyra, Zostera marina, or any combination thereof.

6. The system of claim 1, wherein the growth medium has a pH ranging from 2 to 11.

7. The system of claim 1, further comprising:
a pH sensor that measures the pH of the growth medium; and
a controller communicatively coupled to the pH sensor, wherein the controller monitors the pH of the growth medium and titrates the acid stream to maintain a predetermined pH or pH program in the growth medium.

8. The system of claim 1, further comprising:
an optical sensor that measures one or more optical properties of the growth medium; and
a controller communicatively coupled to the optical sensor, wherein the controller monitors the one or more optical properties of the growth medium and adjusts illumination of the growth medium and/or chemistry of the growth medium in response to the one or more optical properties.

9. A method for coupling electrochemical marine carbon capture with photosynthesis, the method comprising:
using an electrochemical cell configured to be in fluid communication with a saline water source comprising saline water, wherein the electrochemical cell converts the saline water into (i) a base stream comprising hydroxide ions, (ii) an at least partially deionized water stream, and (iii) an acid stream comprising hydrogen ions; and
inputting the acid stream into a biomass cultivation unit comprising a photosynthetic organism and a growth medium for the photosynthetic organism, wherein the acid stream catalyzes CO₂ release from the growth medium to accelerate growth of the photosynthetic organism relative to growth without the acid stream and facilitates CO₂ storage by the photosynthetic organism.

10. The method of claim 9, further comprising transporting contents of the acid stream to the biomass cultivation unit in one or more batches prior to inputting the acid stream into the biomass cultivation unit.

11. The method of claim 9, wherein inputting the acid stream into the biomass cultivation unit comprises fluidly coupling the acid stream with the biomass cultivation unit.

12. The method of claim 9, wherein converting the saline water into the base stream and the acid stream comprises inputting the saline water into a bipolar membrane electrodialysis system comprising a plurality of membranes.

13. The method of claim 12, wherein converting the saline water into the base stream and the acid stream comprises operating the bipolar membrane electrodialysis system in a constant potential mode or a constant current mode.

14. The method of claim 9, wherein the photosynthetic organism comprises a microalgal species, a macroalgal species, a filamentous algal species, a phytoplankton species, a plant species, or a combination thereof.

15. The method of claim 9, wherein the growth medium has a pH ranging from 2 to 11.

16. The method of claim 9, further comprising monitoring the pH of the growth medium and titrating the acid stream to maintain a predetermined pH or pH program in the growth medium.

17. The method of claim 9, further comprising monitoring one or more optical properties of the growth medium and adjusting illumination of the growth medium and/or chemistry of the growth medium in response to the one or more optical properties.

18. A biomass cultivation unit configured to facilitate CO₂ capture, the biomass cultivation unit comprising:
a growth medium;
a photosynthetic organism; and
an acid stream in fluid communication with the growth medium, the acid stream comprising hydrogen ions derived from electrochemical treatment of saline water, wherein the acid stream catalyzes CO₂ release from the growth medium for use by the photosynthetic organism, and wherein the acid stream accelerates growth of the photosynthetic organism relative to growth without the acid stream.

19. The biomass cultivation unit of claim 18, wherein the biomass cultivation unit is in fluid communication with an electrochemical cell configured to be in fluid communication with a saline water source, wherein the electrochemical cell converts saline water from the saline water source into (i) a base stream comprising hydroxide ions, (ii) an at least partially deionized water stream, and (iii) the acid stream.

20. The biomass cultivation unit of claim 18, further comprising:
a pH sensor that measures a pH of the growth medium; and
a controller communicatively coupled to the pH sensor, wherein the controller monitors the pH of the growth medium and titrates the acid stream to maintain a predetermined pH or pH program in the growth medium.
